(19) United States
(12) Patent Application Publication
(10) Pub. No.: US 2026/0258485 A1
(43) Pub. Date: Sep. 3, 2026

(54) METHODS AND KITS FOR IN SITU AMPLIFICATION OF FIXED CELLS

(71) Applicants: Kerry Geiler-Samerotte, Scottsdale, AZ (US); Kara Schmidlin, Mesa, AZ (US); Parker Crossland, Phoenix, AZ (US); Leandra Brettner, Mesa, AZ (US)

(72) Inventors: Kerry Geiler-Samerotte, Scottsdale, AZ (US); Kara Schmidlin, Mesa, AZ (US); Parker Crossland, Phoenix, AZ (US); Leandra Brettner, Mesa, AZ (US)

(73) Assignee: ARIZONA BOARD OF REGENTS ON BEHALF OF ARIZONA STATE UNIVERSITY, Scottsdale, AZ (US)

(21) Appl. No.: 19/553,141

(22) Filed: Feb. 27, 2026

Related U.S. Application Data
(60) Provisional application No. 63/765,359, filed on Feb. 28, 2025.

(51) Int. Cl. C12Q 1/686; C12Q 1/34; C12Q 1/48; C12Q 1/6816; C12Q 1/6841; C12Q 1/6853; C12Q 1/6876

(52) U.S. Cl. CPC C12Q 1/686; C12Q 1/34; C12Q 1/48; C12Q 1/6816; C12Q 1/6841; C12Q 1/6853; C12Q 1/6876; C12Y 207/07007; C12Y 302/01039

(57) ABSTRACT

A method that harnesses the cell membrane as a natural reaction chamber for DNA amplification, enabling optional downstream sequencing and optional downstream cell sorting is described herein. Unlike traditional single-cell techniques, methods herein utilize a strand-displacing, isothermal polymerase to amplify DNA within fixed, permeabilized cells while maintaining the cell's structural integrity. This approach overcomes challenges associated with both typical single-cell DNA sequencing and hindrances encountered when previously attempting to sequence genetic material from fixed microbial cells, allowing amplification of genomic regions up to 100 kb and optional downstream sequencing of whole genomes from diverse cell types, including S. cerevisiae, B. subtilis, and E. coli. Additionally, methods herein (e.g., FISA) can optionally be adapted for targeted DNA enrichment using biotinylated primers and for fluorescence-based cell sorting.

### CROSS-REFERENCE TO RELATED APPLICATIONS

This application is a non-provisional and claims benefit of U.S. Provisional Application No. 63/765,359 filed Feb. 28, 2025, the specification of which is incorporated herein in its entirety by reference.

### STATEMENT REGARDING FEDERALLY SPONSORED RESEARCH OR DEVELOPMENT

This invention was made with government support under Grant No. R35 GM133674 awarded by National Institutes of Health and Grant No. 2119963 awarded by National Science Foundation. The government has certain rights in the invention.

### FIELD OF THE INVENTION

The present invention provides methods and kits for labeled intracellular DNA amplification in such a way as to allow sequencing DNA from a target cell population. Labeling may be performed using fluorescence or other methodologies, and sequencing is facilitated by biotinylation of amplified DNA.

### BACKGROUND OF THE INVENTION

Single-cell DNA sequencing has the potential to transform the ability to study the effects of genetic heterogeneity in populations in experimental evolution, development, and disease. However, a major bottleneck that limits many single-cell ‘omics technologies is the reliance on physical cell isolation which constrains scalability. When performing single-cell genomics, this often restricts studies to hundreds or even dozens of cells. Additionally, such techniques can require time-consuming labor, specialized expertise, and/or costly instrumentation which limits accessibility. While most single-cell DNA methods sort cells into vessels to perform amplification, ironically, each cell's genetic material is naturally compartmentalized by its membrane, providing a built-in reaction chamber. Using the cell as its own reaction container has been extensively harnessed for single-cell transcriptomics. However, intracellular DNA-based applications have not been widely implemented due to several technical challenges, including the hurdle of amplifying DNA in formaldehyde-fixed cells in such a way that allows that DNA to be sequenced.

A significant challenge in intracellular DNA amplification is the need to fix cells with formaldehyde before the in situ reactions. Fixing with formaldehyde stabilizes the cell and the genetic material inside, but the crosslinks that are generated are frequently cited as obstacles to efficient DNA amplification and can hinder sequencing. While some studies suggest that formaldehyde crosslinks can be reversed such that they do not severely inhibit amplification, the concern remains an issue in very recent literature and has driven efforts to perform FISH and FACS in live cells instead. While short, engineered DNA sequences (15 bp) and RNA transcripts have been successfully amplified or reverse transcribed in fixed cells, the ability to sequence intracellularly amplified native genomic DNA from formaldehyde-fixed cells remains a heretofore unmet need.

Here, the present invention features in situ amplification (including, but not limited to) fluorescent in situ amplification (FISA)), a novel method that addresses existing challenges and enables intracellular DNA amplification in cell suspension which unlocks optional downstream applications such as cell sorting based on signal (e.g., fluorescent, biotinylation, etc.) and/or optional downstream DNA sequencing. A key innovation of the in situ amplification of the present invention (including, but not limited to, FISA) is the use of an isothermal polymerase which preserves cell membrane integrity by omitting the need for temperature cycling and likely overcomes formaldehyde crosslinks through its strand-displacing activity. Another important aspect of the invention is the optional use of biotinylated primers that allowed intracellularly amplified DNA to be recovered and washed for downstream sequencing. Another important aspect of the invention is the optional use of reagents that make any intracellularly amplified DNA fluoresce, such that cells containing it can be isolated from other cells. Using in situ amplification techniques of the present invention, successful amplification and sequencing were performed, of target genes up to 3 kb and also whole genomes, in diverse organisms, including S. cerevisiae, B. subtilis, and E. coli. Finally, it was demonstrated using the present invention that up to 14% of the genome can be intracellularly amplified within a single yeast cell that was sorted (in that particular instance) based on fluorescence intensity.

In sum, the in situ amplification techniques of the present invention (including, but not limited to, FISA) provide robust and versatile methods for intracellular DNA amplification, which paves the way for a wide array of optional downstream applications, including high-throughput single-cell sequencing, rare cell genomics, and genotype-based sorting. This approach has the potential to advance the capabilities of single-cell ‘omics, with broad potential applications in fields such as microbial ecology, cancer genomics, and evolutionary biology.

### BRIEF SUMMARY OF THE INVENTION

It is an objective of the present invention to provide methods and kits that allow for intracellular amplification of DNA within fixed cells, in such a way as the DNA from target cell types or genotypes can be sequenced, as specified in the independent claims. Optionally, the intracellularly-amplified DNA may be used for downstream applications, including but not limited to sequencing. Embodiments of the invention are given in the dependent claims. Embodiments of the present invention can be freely combined with each other if they are not mutually exclusive.

In some embodiments, the present invention features a method of intracellularly amplifying DNA within a cell. In some embodiments, the method comprises a) fixing and permeabilizing a liquid sample comprising cells, b) amplifying DNA within the fixed and permeabilized cells by incubating the cells in a solution comprising biotinylated primers, an isothermal polymerase and 5-ethynyl-2′-deoxyuridine (EdU), and c) incubating the cells with a fluorescent azide for a period of time; wherein the fluorescent azide selectively binds to the plurality of EdUs, thereby producing fluorescent cells. In some embodiments, the amplified DNA comprises a plurality of EdUs incorporated therein. In other embodiments, the method comprises a) fixating and permeabilizing a liquid sample comprising cells and b) amplifying DNA within the fixed and permeabilized cells by incubating the cells in a solution comprising biotinylated primers, an isothermal polymerase and fluorescent dNTPs. In some embodiments, the amplified DNA comprises a plurality of fluorescent dNTPs incorporated therein, thereby producing fluorescent cells. In some embodiments, amplification occurs without the use of fluorescence. In some embodiments, cell labeling or DNA labeling may occur via biotinylation, bioluminescence, mass cytometry, radioisotope labeling, use of quantum dots, colorimetric or enzymatic staining, or similar techniques. In some embodiments, intracellularly amplified DNA is labeled with biotin to enable downstream purification and sequencing.

In other embodiments, the present invention comprises a kit for intracellularly amplifying DNA within a cell. In some embodiments, the kit comprises a fixative agent, permeabilization agent, one or more nucleic acid primers that are optionally biotinylated, an isothermal polymerase, and nucleotide analogue (e.g., 5-ethynyl-2′-deoxyuridine (EdU)). Nucleotide analogues may optionally be fluorescent. In other embodiments, the kit comprises a fixative agent, permeabilization agent, one or more nucleic acid primers that are optionally biotinylated, an isothermal polymerase, and dNTPs (which may optionally be fluorescent). As used herein, the term “fluorescent dNTPs” refers to and may include fluorescent nucleotide analogues, including but not limited to EdU.

One of the unique and inventive technical features of the present invention is the use of an isothermal polymerase, and, in some embodiments, biotinylated primers and fluorescent EdUs (5-ethynyl-2′-deoxyuridines). Without wishing to limit the invention to any theory or mechanism, it is believed that these technical features of the present invention advantageously allow for sequencing of DNA that was amplified within a fixed cell. The cell membrane, nuclear envelope (if a eukaryotic cell), and/or other cell membranes serve as natural, self-contained “reaction vessels” for intracellular DNA amplification. Furthermore, this advancement constitutes a surprising result: previously, in order to study DNA, DNA has been extracted prior to amplification. Counterintuitively, the present invention instead amplifies DNA intracellularly before optionally extracting it for optional downstream processing, for example, sequencing. Furthermore, the present invention also allows for the performance of optional “click chemistry” reactions on nucleic acids as performed intracellularly, thereby producing optional intracellular fluorescent signals, made possible by the present invention's ability to perform intracellular nucleic acid amplification. Further still, the present invention's ability to facilitate intracellular nucleic acid amplification also facilitates the optional sorting of cells after nucleic acid amplification. Previously, because nucleic acid amplification has traditionally had to have been performed extracellularly, sorting of intact cells after nucleic amplification has not been possible, because, in order to perform amplification, the cells would first have to be lysed, or otherwise compromised in order to extract the nucleic acids to an extracellular location for amplification. With the present invention, it is now possible to sort intact cells after nucleic acid amplification, since said amplification is performed intracellularly, thereby preserving said cells for sorting (or other downstream applications) after amplification has occurred. None of the presently known prior references or works have the unique inventive technical features of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIGS. 1A-1D show a schematic of the intracellular DNA amplification workflow (in this case specifically, a FISA workflow). This robust and flexible protocol can be altered at each step based on the cell type being amplified and the intended downstream application. FIG. 1A shows fixation to stabilize the cell which needs to remain intact during the entire amplification reaction. It can vary by organism, for example, 4% formaldehyde is best for yeast and 70% ethanol works better for some bacteria. FIG. 1B shows permeabilization which allows reagents to diffuse inside cells. Yeast are treated with zymolyase and triton-X, while some bacterial membranes become permeable during ethanol fixation. FIG. 1C shows reaction reagents including phi29, dNTPs (optionally, in some cases, fluorescent dNTPs), and optionally biotinylated primers are diffused into the cell with help from the crowding agent sorbitol. See methods in later-provided Examples for detailed master mix recipe. FIG. 1D shows DNA amplification occurs via strand displacement.

FIGS. 2A-2B show targeted amplification and sequencing of specific genomic regions from different single celled organisms. FIG. 2A shows gel electrophoresis of amplified DNA fragments targeting different genomic loci. BC: barcode region (~300 bp), SOD1 (~450 bp), 16S rRNA (~1 kb), and PDR3 (~3 kb). Bands corresponding to the expected fragment sizes are highlighted with boxes. FIG. 2B shows sequencing coverage plots for SOD1, 16S rRNA, and PDR3, which show the read depth across each amplified region. Coverage varies across each fragment, but most bases are covered over 15×. Because amplification is carried out using phi29 polymerase with gene-specific primers in a strand-displacing, isothermal reaction, amplification proceeds unidirectionally from the primer rather than symmetrically from both ends.

FIGS. 3A-3B show Whole Genome intracellular Amplification and Sequencing in S. cerevisiae and E. coli. FIG. 3A shows S. cerevisiae whole genome amplification and sequencing results. Left: Gel electrophoresis image showing amplified DNA from S. cerevisiae, with a distribution of fragment sizes. Right: Read depth across the S. cerevisiae genome, with coverage visualized along the chromosome in a gradient. FIG. 3B shows E. coli whole genome amplification and sequencing results. Left: Gel electrophoresis image of amplified E. coli DNA, with a prominent high-molecular-weight smear and a boxed region highlighting a specific fragment size. An asterisk (*) indicates an additional band corresponding to the size of a 16S amplicon, as 16S primers were added alongside random hexamers in this reaction. This suggests that multiple primer types can be used within the same reaction. Right: Read depth across the E. coli genome, showing sequencing coverage.

FIGS. 4A-4D show fluorescently labeled Intracellular Genome Amplification, followed by single cell sorting of only the most fluorescent cells, followed by independent collection and sequencing of the intracellularly amplified DNA from 4 single cells. FIG. 4A shows a schematic representation of intracellular genome amplification. Control cells (left) do not undergo amplification, while treated cells (right) contain newly synthesized DNA. In some embodiments, the DNA is stained with a fluorescent DNA binding dye such as EvaGreen®, and fluorescence intensity is measured by flow cytometry, revealing increased fluorescence in amplified cells compared to controls. Amplified cells are then sorted for sequencing; in this experiment, 4 cells were sorted. FIG. 4B shows biotinylated primers enable selective extraction of newly amplified DNA using magnetic streptavidin beads, followed by multiple washes to purify the sample. FIG. 4C shows genome coverage analysis of four individual cells mapped to S. cerevisiae chromosomes. Each colored segment represents a covered region for a specific cell. FIG. 4D shows a summary of genome coverage for each sequenced cell. The percentage of the genome covered is highest in Cell 1 (13.62%) and lowest in Cell 4 (2.92%).

FIG. 5 shows a potential schematic of workflow and optional downstream applications of the present invention (including, but not limited to, FISA). Fixed cells undergo intracellular amplification, where they are permeabilized to allow enzymatic amplification of their genomic DNA. The amplified DNA incorporates nucleotides (which, in some embodiments may be modified nucleotides), enabling downstream applications. One application involves optional integration with DNA barcoding, allowing unique sequence tagging for lineage tracing or multiplexed analysis. Another application is an optional fluorescence-based assay to detect the presence or absence of specific genes based on fluorescence signals. Additional potential applications (indicated by “Other Applications”) may include further genomic, transcriptomic, or functional analyses.

FIGS. 6A-6B shows that washing the cells and/or streptavidin-bound DNA removes any DNA that is not biotinylated and intracellularly amplified, ensuring selective retention of the amplified DNA following intracellular amplification and recovery in those embodiments of the present invention that utilize biotinylation. The schematics illustrate intracellular amplification within fixed cells, followed by washing steps to remove any extracellular DNA/cell debris generated from lysis (Left). Agarose gel electrophoresis of DNA recovered after successive washes is also depicted. The decreasing signal across washes indicates progressive removal of non-amplified (native) DNA (Right). Wash steps remove extracellular and non-biotinylated DNA during the intracellular DNA amplification workflow. Note that, alternatively, in some instances, the “pre-wash” referred to in both FIG. 6A and FIG. 6B may be referred to as “wash 1,” in which case the “wash 1” in both FIG. 6A and FIG. 6B is referred to as “wash 2,” and “wash 2” is referred to as “wash 3.” FIG. 6A shows that to test whether extracellular DNA is effectively removed before lysis, biotinylated E. coli 16S amplicons (1 ng/μL [lane a] or 10 ng/μL [lane b]) were spiked into S. cerevisiae samples pre-Phi29 amplification. This is an enormous amount of extracellular DNA; 10 ng corresponds to the genomic equivalent of nearly all the yeast cells in the sample (1 million cells). This spiked-in biotinylated DNA is intended to mimic any extracellular DNA that would have been amplified with biotinylated primers by phi29. After the 16-hour incubation step during which intracellular amplification occurs, cells were subjected to two sequential pre-lysis washes. Supernatants before each wash were cleaned up using size selection beads and underwent PCR-amplification using custom E. coli 16S-specific primers designed to generate a ~1 kb amplicon. The gel reveals a decreasing signal across washes, which confirms that extracellular DNA is removed by pre-lysis washes. Lane “−” is a negative control reaction with no DNA added. FIG. 6B shows that to test whether a cell's native genome is effectively removed after lysis and bead binding, non-biotinylated (mimicking a cell's native genome) E. coli 16S amplicons (1 ng/μL [lane a] or 10 ng/μL [lane b]) were spiked into cell lysates post lysis. The lysates were bound to streptavidin beads in accordance with the INgen protocol. Bead-bound DNA underwent two washes. Supernatants before each wash step were cleaned up with size selection beads and PCR-amplified as in FIG. 6A. The gel reveals a reduction in signal across washes, which indicates that non-biotinylated DNA is efficiently removed from bead-bound samples. Lane “−” is a negative control with no DNA added.

FIGS. 7A-7F shows a schematic of the intracellular DNA amplification (INgen) workflow. INgen is another technique of the present invention, and an alternative to FISA and similar other techniques of the present invention. This robust and flexible protocol can be altered at each step based on the cell type being amplified. FIG. 7A shows that fixation stabilizes the cell, which needs to remain intact during the entire amplification reaction. The formaldehyde concentration selected varies by organism: for example, 4% formaldehyde for yeast and 70% ethanol for bacteria. FIG. 7B shows that permeabilization allows reagents to diffuse inside cells. Yeast are treated with zymolyase and triton-X, while bacterial membranes become permeable during ethanol fixation. FIG. 7C shows that reaction reagents, including phi29, dNTPs, and primers, are diffused into the cell by the crowding agent sorbitol. See Example 5 of the present application for the detailed master mix recipe. FIG. 7D shows that intracellular strand displacement amplification (ISDA) proceeds isothermally via primer annealing, polymerase extension, and strand displacement, generating concatemeric DNA products within each intact cell. FIG. 7E shows that cell washing removes extracellular DNA and unincorporated reagents while maintaining the integrity of amplified cells. FIG. 7F shows that cell lysis releases amplified DNA, which contains incorporated biotin. The biotinylated DNA is bound to magnetic streptavidin-coated beads, enabling optional subsequent purification and downstream processing such as sequencing library preparation.

FIGS. 8A-8B show whole-genome amplification and sequencing of E. coli DH5α. FIG. 8A shows agarose gel electrophoresis of amplified E. coli DNA showing a high-molecular-weight smear indicative of successful whole-genome amplification. The boxed region highlights a representative fragment size within the amplification range. The asterisk (*) marks an additional band corresponding to the expected size of a 16S rRNA amplicon, as 16S primers were included alongside random hexamers in the same reaction, demonstrating that multiple primer types can be used concurrently. FIG. 8B shows sequencing coverage across the E. coli DH5α genome. Read depth is shown in orange, coverage exceeds 30× across 99.4% the 4.6-Mb circular chromosome.

FIGS. 9A-9B show controls demonstrating that amplification is specific to intracellular and biotinylated DNA. FIG. 9A shows that, to test whether non-biotinylated DNA binds non-specifically to beads and is retained through washing, E. coli 16S DNA (1 ng [lane a], 10 ng [lane b]) was spiked into lysates after cell lysis. Lysates were incubated with streptavidin beads, the beads were washed in accordance with the INgen protocol (see Example 5), and amplification was performed directly off the beads using E. coli 16S-specific primers. The absence of signal in lanes a and b demonstrates that non-biotinylated DNA does not remain associated with the beads. Lane “−” is a no-DNA negative control. Lanes “+” and “++” are positive controls where 1 ng and 10 ng of E. coli 16S DNA, respectively, were directly amplified using the same primers. FIG. 9B shows that, to test whether extracellular DNA can diffuse into intact cells, biotinylated E. coli 16S DNA (1 ng [lane a], 10 ng [lane b]) was spiked into samples before Phi29 treatment. This is an enormous amount of spiked-in DNA; 10 ng corresponds to the genomic equivalent of nearly all the yeast cells in the sample (1 million cells). After pre-lysis washes, cells were lysed and streptavidin beads were used to bind any biotinylated DNA in the lysate. Amplification off-the-beads shows no signal in lane a but some signal in lane b. These results suggest that extracellular biotinylated DNA does not appreciably diffuse into intact cells at reasonable concentrations. Lanes “−”, “+”, and “++” serve as negative and positive amplification controls as in panel (a).

FIG. 10 shows MinION sequencing shows intracellularly amplified DNA up to 30,000 bp long. The histogram of read lengths was obtained from Oxford Nanopore MinION sequencing of intracellularly amplified DNA. The majority of reads are between 1,000 and 10,000 bp, with a peak around 2,000 bp. A substantial tail of longer reads extends beyond 20,000 bp, with some reads reaching lengths up to ~30,000 bp. These data demonstrate the capability of the intracellular amplification protocol to generate long DNA products suitable for long-read sequencing.

FIG. 11 shows that a library QC of intracellularly amplified DNA from formaldehyde-fixed E. coli cells shows a Mean Read Length of 16,951 bp. Femto Pulse trace of DNA amplified intracellularly from formaldehyde-fixed E. coli cells is depicted. The electropherogram shows a broad distribution of fragment sizes, with a mean read length of 16,951 bp, indicating successful amplification of long DNA molecules.

FIG. 12 shows a library QC of intracellularly amplified DNA from ethanol-fixed E. coli cells shows a Mean Read Length of 16,822 bp. Femto Pulse trace of DNA amplified intracellularly from ethanol-fixed E. coli cells is depicted. The size profile closely resembles that of the formaldehyde-fixed sample, with a mean fragment length of 16,822 bp, supporting robust amplification from both fixation conditions.

FIG. 13 shows coverage across the 16S rRNA gene of Bacillus subtilis following intracellular amplification and sequencing. The coverage plot shows read depth across the length of the B. subtilis 16S rRNA gene. DNA was first amplified intracellularly using gene-specific primers and Phi29 polymerase, followed by lysis and streptavidin bead capture. The bead-bound product was then re-amplified using specific primers and OneTaq polymerase. This off-the-beads product was sequenced, and the resulting reads demonstrate high coverage across the gene, with expected tapering at the 5′ and 3′ ends. These results indicate efficient and relatively uniform amplification of full-length 16S rRNA sequences using this workflow.

### DETAILED DESCRIPTION OF THE INVENTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessarily solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar to or equivalent to those described herein can be used to practice or test the disclosed technology, suitable methods and materials are described below. The materials, methods, and examples are illustrative only and not intended to be limiting.

As used herein, “fixation” or “fixing” refers to the process of chemically stabilizing organic, inorganic, or a combination of organic and inorganic molecules through the use of reagents, known as “fixatives”. Exemplary fixatives for the present disclosure include, but are not limited to, formaldehyde, formaldehyde derived from paraformaldehyde, formalin, phosphate buffered formalin, formal calcium, formal saline, zinc formalin, alcoholic formalin, glutaraldehyde, other organic aldehydes, methanol, ethanol, isopropanol, or other organic alcohols, or solutions containing organic alcohols or aldehydes.

As used herein, “permeabilization” or “permeabilizing” refers to the process of introducing openings into barriers to allow the penetration of desired molecules past the aforementioned barrier. In some embodiments, the barrier comprises a cell membrane, and or a cell wall. In some embodiments, permeabilization is performed by, for example, enzymes on biological membranes. Exemplary enzymes for permeabilization of biological membranes include but are not limited to proteinase K and zymolyase. In some embodiments, permeabilization is performed by, for example, detergents on biological membranes.

As used herein, “amplification” refers to the process of semi-conservatively replicating nucleic acid strands by enzyme-catalyzed extension. Exemplary enzymes for amplification of nucleic acids in the current disclosure include, for example, nucleic acid polymerases. In some embodiments, an isothermal polymerase is used to amplify nucleic acids. In some embodiments, amplification is carried out with a high-fidelity polymerase, such as Q5, with the technique known as the polymerase chain reaction (PCR). Amplification can be performed with natural and non-natural nucleotide bases, ribonucleotide bases or deoxyribonucleotide bases, labeled nucleotide bases, and the like.

As used herein, “isothermal amplification” describes amplification of DNA targets without heat denaturation of DNA. In contrast, polymerase chain reaction (PCR) requires cycling through different temperatures for denaturation, hybridization, and extension. Isothermal amplification may be preceded by a higher temperature hybridization step that does not denature the DNA target. Exemplary polymerases useful for isothermal amplification are referred to herein as isothermal polymerases, and include, but are not limited to phi29 polymerase, Klenow exo-DNA Polymerase I, Bsu polymerase, Bst polymerase, Bsm polymerase.

As used herein, “sequencing” refers to the sequencing of nucleic acids. Sequencing of nucleic acids may be accomplished using, by way of example but not by way of limitation, Sanger sequencing, or next-generation sequencing. It should be noted that, in general, sequencing is an optional step in the present invention, which optionally occurs downstream of intracellular amplification.

As used herein, the term “random hexamer” or “random hexanucleotide” refers to a region of six nucleotides in length comprising sequences that are synthesized at random. The purpose of random hexamers is, in most applications, to bind complementarily to nucleotide sequences of unknown identity. Thus, because random hexamers theoretically cover all possible sequence permutations for a hexameric (6-member) nucleotide, they are likely to bind at many positions to nucleotides of any sequence. It should be understood, however, that a key feature of random hexamers is not that they are six nucleotides in length, but rather that they have random sequence identity. In other words, for many applications it is possible to provide random pentamers (5-member), heptamers (7-member), or other random sequences in place of hexamers. In some embodiments, a random hexamer comprises a part of, or a portion of a larger oligonucleotide, such as an oligonucleotide primer.

As used herein, “crowding agent” refers to compounds that decrease the solvent available to macromolecules, thereby increasing the relative concentration of said macromolecules and altering their properties. In some applications, crowding agents have the effect of increasing enzyme activity and accelerating reactions resulting in faster and potentially more specific assays. In some embodiments, crowding agents may include one or more polyethylene glycol (PEG), polyethylene glycol 8000 (PEG-8000), trehalose, and sorbitol. In some embodiments crowding agents may include ficoll or dextrans.

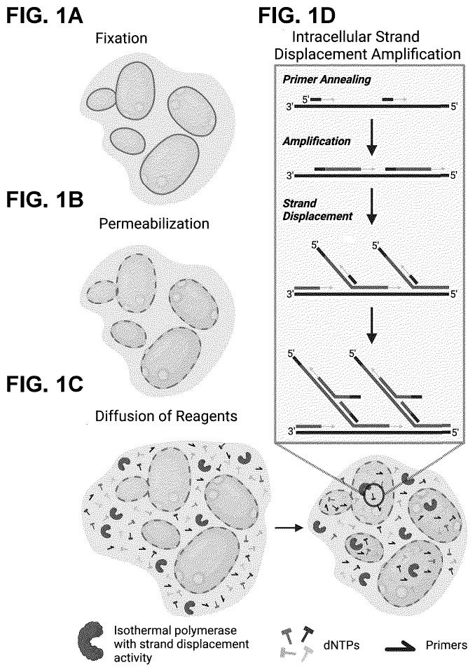

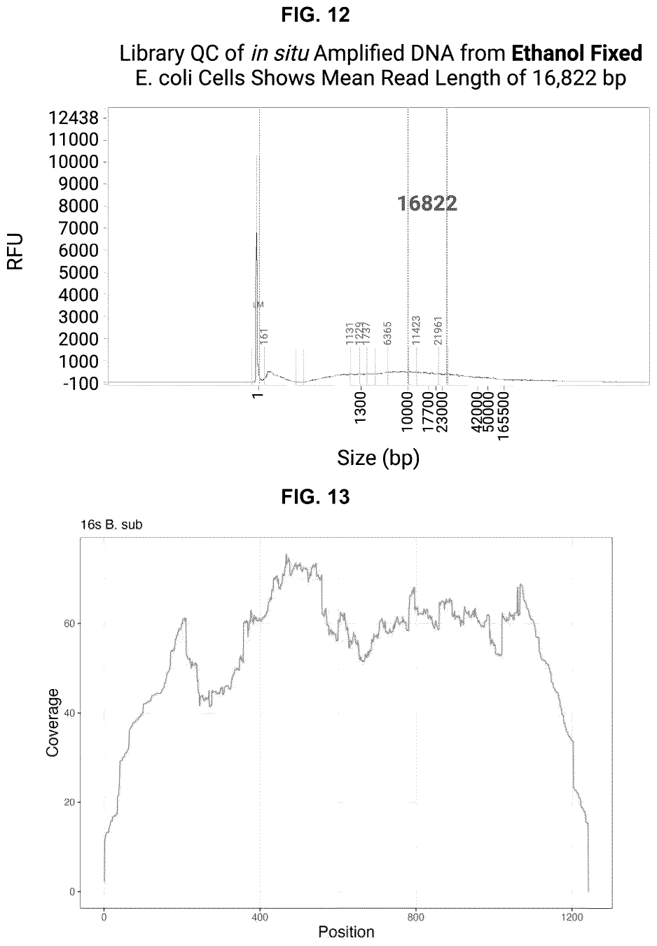

Referring now to FIGS. 1A-13, the present invention features novel methods that enable intracellular DNA amplification, including but not limited to a gene of interest, using a thermostable polymerase, with dNTPs (including but not limited to EdU (5-ethynyl-2′-deoxyuridine)) incorporated into newly synthesized DNA. This allows for, in some embodiments, DNA to be fluorescently labelled via click chemistry (e.g., Click-iT®) reaction, enabling detection by flow cytometry. The methods described herein may be used alone or in combination with other protocols (e.g., FISH). In some embodiments, the methods described herein enable robust amplification and optional downstream sequencing of single genes and entire genomes while preserving cellular integrity and minimizing false positives. In some embodiments, the present invention utilizes fluorescent labeling technology. The versatile methods provided herein offer powerful tools for studying rare genotypes and genetic heterogeneity in contexts ranging from evolution to medicine.

In some embodiments, the present invention features a method of intracellularly amplifying DNA within a cell. In some embodiments, the method comprises a) fixating and permeabilizing a liquid sample comprising cells, b) amplifying DNA within the fixed and permeabilized cells by incubating the cells in a solution comprising an isothermal polymerase and (optionally) 5-ethynyl-2′-deoxyuridine (EdU), and c) (optionally) incubating the cells with a fluorescent azide for a period of time; wherein the fluorescent azide selectively binds to the plurality of EdUs, thereby (optionally) producing fluorescent cells. In some embodiments, the amplified DNA comprises a plurality of EdUs incorporated therein.

In other embodiments, the method comprises a) fixating and permeabilizing a liquid sample comprising cells and b) amplifying DNA within the fixed and permeabilized cells by incubating the cells in a solution comprising an isothermal polymerase and (optionally) fluorescent dNTPs. In some embodiments, the amplified DNA comprises a plurality of fluorescent dNTPs incorporated therein, thereby (optionally) producing fluorescent cells.

In further embodiments, the method comprises a) fixing and permeabilizing a liquid sample comprising cells and amplifying DNA within the fixed and permeabilized cells by incubating the cells in a solution comprising: an isothermal polymerase and (optionally) a fluorescent DNA binding dye such as EvaGreen®. In some embodiments, the fluorescent DNA binding dye binds to the amplified DNA, thereby (optionally) producing fluorescent cells.

In some embodiments, isothermal polymerase comprises a phi29 polymerase. In other embodiments, the isothermal polymerase is a Bst polymerase. In some embodiments, the isothermal polymerase is another isothermal polymerase.

Without wishing to limit the present invention to any theory or mechanism it is believed that the use of an isothermal polymerase that (1) amplifies DNA at low temperatures that do not damage cell membranes and (2) can contend with formaldehyde crosslinks via its strand displacing ability provides the present invention with the ability to amplify DNA intracellularly.

In some embodiments, the methods described herein allow for successful intracellular amplification and (optional) sequencing of target genes up to 3 kb in size and/or the whole genomes of species with different types of cell membranes including fungal cells (S. cerevisiae) gram-positive bacteria (B. subtilis) and gram-negative bacteria (E. coli). In some embodiments, formaldehyde crosslinks do not disrupt the isothermal polymerase by demonstrating that the amplified pieces of DNA are very long (up to 100 KB).

In some embodiments, the solution further comprises enzymes, dNTPs (which, optionally, may be fluorescent dNTPs), a buffer, and one or more nucleic acid primers.

In some embodiments, the nucleic acid primers comprise biotinylated primers. In other embodiments, the nucleic acid primers comprise sequence-specific primers. In some embodiments, the sequence-specific primers are biotinylated. In certain embodiments, the nucleic acid primers include random oligonucleotide primers. In some embodiments, the random oligonucleotide primers are biotinylated.

In some embodiments, the aforementioned methods further comprise separating fluorescent cells. In other embodiments, the aforementioned methods further comprise separating fluorescent single cells. In other embodiments, the fluorescent cells are separated by flow cytometry, microscopy, or microfluidics. In other embodiments, the cells to be separated are labeled using an alternative labeling technique, including but not limited to biotinylation.

In some embodiments, the aforementioned methods further comprise lysing the separated cells. In other embodiments, the aforementioned methods further comprise lysing the separated cells and amplifying the DNA or other genetic components of said cells. In some embodiments, amplifying the DNA in the separated cells comprises introducing a second solution comprising enzymes, dNTPs (which, optionally, may be fluorescent dNTPs), a buffer, and one or more nucleic acid primers.

In other embodiments, the aforementioned methods optionally comprise sequencing the separated cells.

Additionally, without wishing to limit the present invention to any theory or mechanism, it is believed that the methods described herein contend with the challenge of amplifying DNA post cell lysis. Lysis chemicals can be harsh and interfere with DNA amplification. Formaldehyde may exacerbate this issue by requiring stronger lysis reagents. But since the first amplification is performed intracellularly (before cell lysis), one “starts out” with more template DNA post lysis, which may aid the amplification reactions that occur post-lysis and prior to sequencing. Further, the optional use of biotinylated primers allows the lysis solution to be washed away, facilitating subsequent amplification and DNA sequencing reactions.

Biotinylation is one of several optional implementations of the present technology that allow for more diverse downstream applications. In one implementation, the primers used to intracellularly amplify DNA are biotinylated. This allows intracellularly amplified DNA to be bound to streptavidin beads after cell lysis (because biotin binds streptavidin). Any unbound DNA, which represents native genomic DNA or contaminants, can then be washed away. This implementation can be useful when there is only a small amount of template DNA (i.e. single-cell sequencing) because in that case the template is more likely to be overwhelmed by such contaminants. The provided methods and techniques of intracellularly amplifying DNA with biotinylated primers allows such contaminants to be washed away, leaving only the intracellularly-amplified pieces of interest.

Another optional implementation is the use of Click-It® chemistry to make the intracellularly amplified DNA fluorescent. In this implementation, EDUs, which are typically fed to growing cells, are instead diffused into dead, fixed cells. Here, they are incorporated into intracellularly amplified DNA. Incubation of these cells with an azide bound fluorophore (e.g., Alexa Fluor®) results in the cells with intracellularly amplified DNA giving off a fluorescent signal. In some embodiments, the azide bound fluorophore is a fluorescent molecule or other moiety (e.g., macromolecule) that has been chemically modified with an azide group. In some embodiments, the azide bound fluorophore binds, via its azide group, to an alkyne group present on a separate molecule or macromolecule (including but not limited to fluorescent dNTPs). In some embodiments, binding of the azide bound fluorophore's azide group to the alkyne group (together with binding to a catalyst) produces a 1,2,3-triazole ring, thereby covalently linking the fluorophore (bound to the azide group) to the alkyne, and thereby in turn to the attached molecule or macromolecule (e.g., fluorescent dNTPs). In some embodiments, the fluorescent dNTP is EdU. In some embodiments, the azide bound fluorophore is Alexa Fluor®. This approach of detecting which cells possess intracellularly amplified DNA is superior to one that involves a probe because in the method of the present invention described above, only the intracellularly amplified DNA gives off a fluorescent signal.

In sum, the present invention provides techniques, kits, and the like, to intracellularly amplify DNA, which allows for optional downstream DNA sequencing; this ability is ripe for still other downstream applications, including reactions involving barcoding, single-cell sequencing, sorting, rare cell genomics, etc.

In other embodiments, the present invention comprises a kit for intracellularly amplifying DNA within a cell. In some embodiments, the kit comprises a fixative agent, permeabilization agent, one or more nucleic acid primers that may optionally be biotinylated, an isothermal polymerase, and nucleotide analogue (e.g., 5-ethynyl-2′-deoxyuridine (EdU)) and optionally a fluorescent modifier (e.g., azide bound Alexa Fluor®). In other embodiments, the kit comprises a fixative agent, permeabilization agent, one or more nucleic acid primers, an isothermal polymerase, and optionally fluorescent dNTPs.

In some embodiments, the fixative agents comprise formaldehyde and/or ethanol. In some embodiments, the permeabilization agent comprises an enzymatic treatment and/or a detergent. In some embodiments, the enzymatic treatment comprises zymolyase. In some embodiments, the detergent comprises Tween-20 or Triton X-100. In some embodiments, the nucleic acid primers comprise biotinylated primers, sequence-specific primers, or random oligonucleotide primers.

In some embodiments, the present invention features a method of intracellularly amplifying DNA within a cell, comprising: a) fixing and permeabilizing a sample comprising cells; b) amplifying DNA within the fixed and permeabilized cells by incubating the cells in a solution comprising: i) an isothermal polymerase; ii) dNTPs; iii) one or more modified nucleic acid primers; wherein the isothermal polymerase incorporates the one or more dNTPs into the amplified DNA, wherein the amplified DNA comprises one or more extended modified nucleic acid primers.

In some embodiments, the isothermal polymerase is a phi29 polymerase or a Bst polymerase.

In some embodiments, the solution further comprises at least one of: enzymes and a buffer.

In some embodiments, the one or more modified nucleic acid primers comprise biotinylated primers. In some embodiments, the one or more modified nucleic acid primers comprise one or more modified sequence-specific primers, one or more modified random oligonucleotide primers, or a combination thereof.

In some embodiments, the present invention features a method of intracellularly amplifying DNA within a cell comprising: a) fixing and permeabilizing a liquid sample comprising cells; and b) amplifying DNA within the fixed and permeabilized cells by incubating the cells in a solution comprising: i) an isothermal polymerase; and ii) fluorescent dNTPs; wherein the isothermal polymerase incorporates one or more fluorescent dNTPs into the amplified DNA, thereby producing fluorescent cells.

In some embodiments, the solution further comprises a fluorescent DNA binding dye; wherein the fluorescent DNA binding dye binds to the amplified DNA, thereby producing fluorescent cells.

In some embodiments, the isothermal polymerase is a phi29 polymerase or a Bst polymerase.

In some embodiments, the solution further comprises at least one of: enzymes, a buffer, or one or more nucleic acid primers.

In some embodiments, the solution further comprises dNTPs.

In some embodiments, the solution further comprises 5-ethynyl-2′-deoxyuridine (EdU) and a fluorescent azide; wherein the method further comprises incubating the cells with a fluorescent azide for a period of time; and wherein the fluorescent azide selectively binds to the one or more EdUs, thereby producing fluorescent cells.

In some embodiments, at least one of the nucleic acid primers comprise at least one of: biotinylated primers, sequence-specific primers, or random oligonucleotide primers.

In some embodiments, the present invention features a kit comprising: a) fixative agents; b) a permeabilization agent; c) one or more modified nucleic acid primers; and d) an isothermal polymerase.

In some embodiments, the kit further comprises at least one of: 5-ethynyl-2′-deoxyuridine (EdU), an azide bound fluorophore, fluorescent dNTPs, or fluorescent DNA binding dye.

In some embodiments, the fixative agents of the kit comprise at least one of: formaldehyde, ethanol, or isopropanol.

In some embodiments, the permeabilization agent of the kit comprises at least one of: an enzymatic treatment and/or a detergent.

In some embodiments, the enzymatic treatment of the kit comprises zymolyase.

In some embodiments, the detergent of the kit comprises Tween-20 or Triton X-100.

In some embodiments, the nucleic acid primers of the kit comprise at least one of: biotinylated primers, sequence-specific primers or random oligonucleotide primers.

### Example 1

The following is a non-limiting example of the present invention. It is to be understood that said example is not intended to limit the present invention in any way. Equivalents or substitutes are within the scope of the present invention.

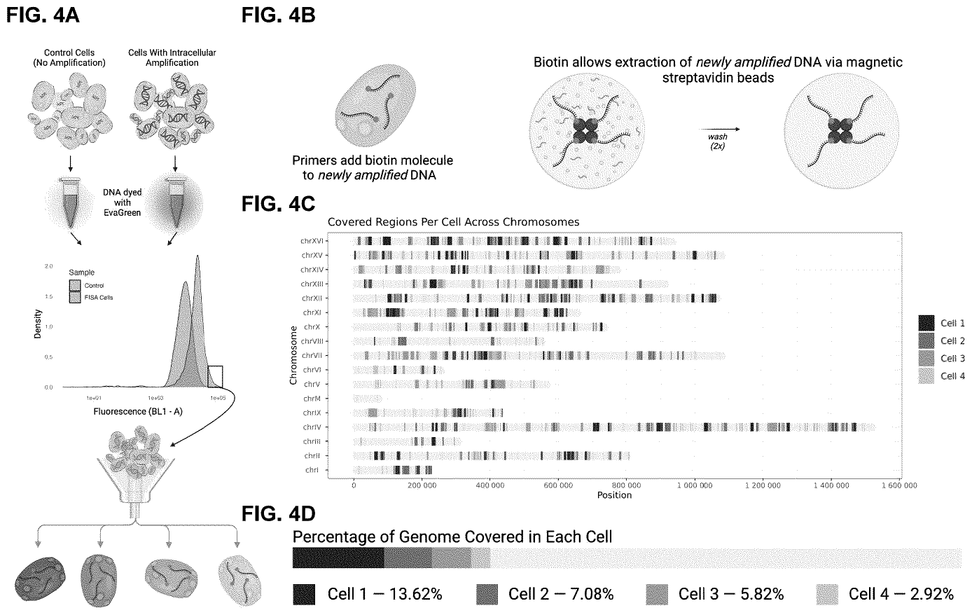

A flexible method for fluorescent in situ Amplification (FISA): The core challenge for downstream applications of intracellular amplification is addressed herein: successfully amplifying DNA within a single cell, labelling it (in this embodiment) with a fluorescent molecule and demonstrating this DNA can be sequenced. The FISA workflow (the subject of this Example 1) consists of five key steps: (1) fixation and permeabilization of cells stabilizes cellular structure and allows reagent access, (2) intracellular DNA amplification using an isothermal polymerase and optionally biotinylated primers, (3) optionally sorting cells based on (in this embodiment) fluorescent intensity, (4) optional cell lysis and isolation of newly synthesized DNA via streptavidin-coated beads, and (5) optional downstream sequencing of intracellularly amplified genomic material (FIG. 1A-1D, FIG. 4A-4D). It is shown that intracellular amplification, sorting and subsequent sequencing of amplified products is not only possible but can be successfully applied in multiple contexts which highlights this method's utility for optional downstream single-cell sequencing applications.

To assess the broad applicability of this method across taxa, intracellular amplification in the eukaryotic model organism Saccharomyces cerevisiae (yeast) and two prokaryotic species, Bacillus subtilis and Escherichia coli was tested. Using species-specific fixation and permeabilization protocols, genomic material was successfully amplified in all three organisms. For yeast, fixation with formaldehyde followed by treatment with zymolyase and triton-X enabled effective permeabilization, while ethanol fixation proved most effective for E. coli and B. subtilis. The results demonstrate that this platform is robust to differences in cell wall composition and fixation requirements, making it broadly applicable across phylogenetically distant organisms.

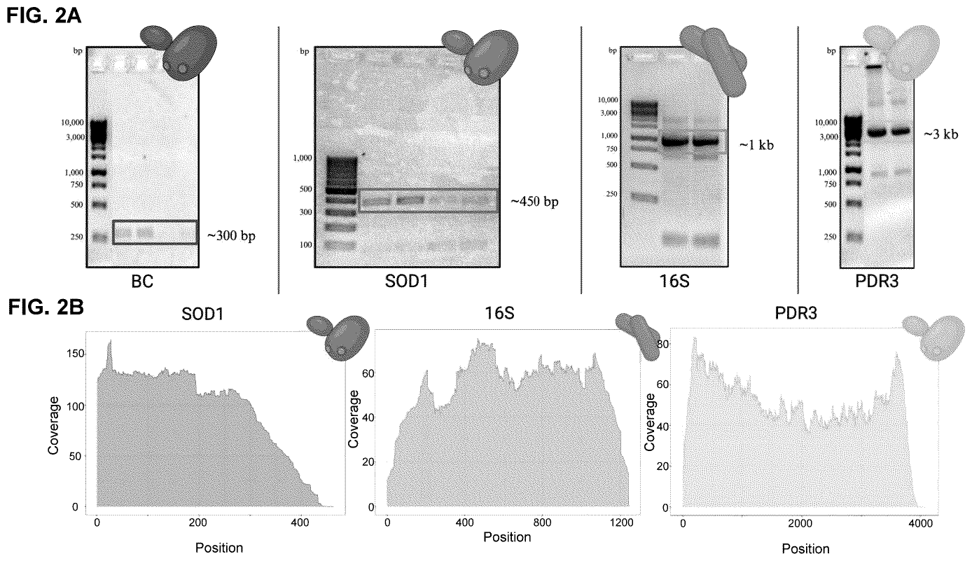

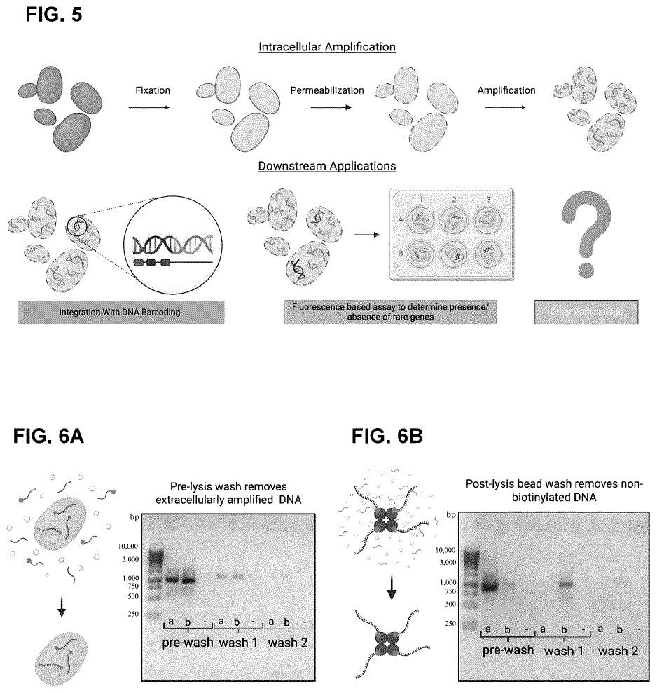

In-situ amplification and sequencing of targeted genomic regions: To evaluate the ability of FISA (the technique of this Example 1) to selectively amplify specific genomic regions, biotinylated primers were designed targeting genes of varying lengths, including three genes from yeast (SOD1, 450 bp; PDR3, 3,000 bp; and an engineered barcode BC, 300 bp) and one gene from bacteria (16S rRNA, 1,000 bp). Cells were incubated with phi29 master mix for 16 hours at 30° C. lysed and newly synthesized DNA amplicons were isolated via streptavidin-coated beads. Once bound to the streptavidin-coated beads and washed three times, a secondary extracellular amplification reaction with the same targeted primers (modified for extracellular amplification) was performed to generate sufficient DNA that is not bound by streptavidin beads for library preparation. Gel electrophoresis analysis confirmed successful targeted amplification, with distinct bands of the expected sizes observed for all tested loci (FIG. 2A). In contrast, amplification reactions performed using the wash (non-bead bound DNA) failed to produce detectable products, even when large amounts of DNA were spiked into the tube prior to washing. This confirms that amplification is specific to intracellularly amplified, biotinylated DNA and not native genomic DNA (FIG. 4A-4D and FIG. 6A-6B).

A wide range of gene lengths were intracellularly amplified, with the smallest being ~300 bp and the largest being ~3000 bp. While amplification of any regions above 3 kb was not attempted, the highly processive nature of phi29 suggests that longer targets can also be amplified. Following size confirmation, fragments were (in this embodiment) prepared for sequencing using the Illumina DNA Flex kit. Targeted genomic regions were successfully sequenced to a minimum depth of 15× which is sufficient for variant calling (FIG. 2B). The ability to target multiple genes across multiple species highlights the flexibility and broad applicability of this method.

Uniform whole genome amplification with random hexamers in diverse cell types: A potential concern with FISA (the technique that is the subject of this Example 1) is whether certain genomic regions might be inaccessible to the isothermal polymerase, particularly in eukaryotic cells where DNA is tightly packed within the nucleus. However, experiments conducted herein suggest this is not a major limitation. To assess whether specific genomic regions are resistant to intracellular amplification, FISA whole genome amplification was performed in fixed yeast and bacterial (E. coli) cells using biotinylated random hexamers. Newly synthesized (i.e. biotinylated) DNA was then isolated from single cells, and additional rounds of PCR were performed using random hexamers to copy these DNA sequences off of the beads, and the resulting pool of DNA was submitted for sequencing.

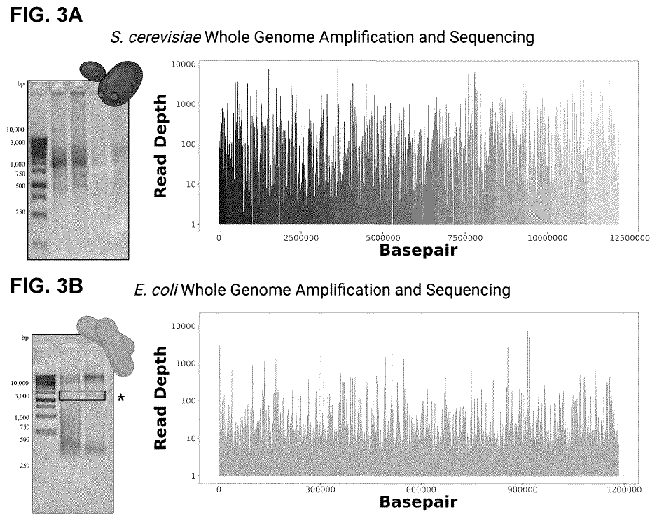

Gel electrophoresis of off-the-beads PCR product revealed smears spanning a broad range of fragment sizes, consistent with successful whole-genome amplification. In yeast, fragment sizes ranged from 200 bp to 50 kb (FIG. 3A), while bacterial genomes displayed similar smearing patterns (FIG. 3B). TapeStation analysis further confirmed that intracellular amplification generated long DNA fragments, with an average size of ~20 kB, and some spanning up to several hundred kilobases, consistent with the expected performance of phi29 polymerase. These results suggest that formaldehyde crosslinking does not significantly inhibit polymerase activity.

Finally, Illumina sequencing of the amplified DNA reveals roughly uniform coverage across the bacterial genome (FIG. 3B). While some variation in sequencing depth was observed, the distribution is consistent with previous studies using isothermal polymerase for whole-genome amplification. Importantly, sequencing of pooled reads recovered nearly the entire E. coli genome (FIG. 3B), confirming FISA's ability to amplify diverse genomic regions without significant bias. These results demonstrate that intracellular amplification is robust across different organisms and genome complexity, validating its utility for whole-genome sequencing applications.

Here it is demonstrated that it is possible to amplify the majority of the genome of S. cerevisiae or E. coli by performing in situ amplification with biotinylated random hexamers (FIG. 3A-3B). From a single in situ PCR reaction which contained ~1 million cells, 24.28% of the yeast genome was covered to an average depth of 26.57 by ~5 million Illumina 150PE reads (FIG. 3A). 98.59% of the E. coli genome was covered to an average depth of 19.81 with the same number of reads (FIG. 3B). While the entire genome is not covered, relatively uniform sequencing coverage was noticed, which indicates that phi29 amplification does not introduce significant bias towards highly transcribed or more accessible genomic regions.

As with targeted genomic amplification, the use (in this embodiment) of biotinylated primers enables selective enrichment of intracellularly amplified DNA, while allowing non-biotinylated DNA and biotinylated DNA outside of a cell to be effectively removed by wash steps. This approach mitigates contamination issues commonly encountered in traditional single-cell genome sequencing, where cells are lysed before amplification, which can result in exogenous DNA being amplified along with the limited amount of starting genomic DNA. By selectively retaining only genomic DNA, the present method improves sequencing accuracy and reliability.

Successful sorting and sequencing of individual cells with intracellularly amplified DNA: To further validate that amplification is occurring in situ, control and test cells were stained with EvaGreen®, a fluorescent dye that binds to all double-stranded DNA, and fluorescence distributions were compared. Indeed, test populations exhibited significantly greater average fluorescence compared to controls, which lacked primers and/or polymerase (FIG. 4A). Since EvaGreen® stains both the native genome and newly made DNA, flow cytometry was used to identify only those test cells exhibiting brighter fluorescence than measured in any control. Of the test cells that were brighter than any control, cells with the greatest BL1-A values were then gated, assuming that higher fluorescence indicated more robust genomic amplification. The fluorescent cells that met both criteria were sorted into individual wells of a 96-well plate for downstream single-cell sequencing. The primary objective was to determine the fraction of the genome amplified within any given single cell and to assess how many single cells must be pooled to recover a complete genome.

Single-cell sequencing was (in this embodiment) then performed on 4 of the 96 sorted single cells. Each cell was lysed using Proteinase K, and the newly synthesized, biotinylated DNA was captured with streptavidin-coated beads and washed several times to remove native genomic DNA (FIG. 4B). For all four cells, the bead-bound DNA was then re-amplified using random hexamers to generate enough starting material (>5 ng/uL) to construct sequencing libraries. This enabled the specific sequencing of only newly synthesized DNA from individual cells specifically, which allows one to then report the percent of the genome this method can amplify per cell.

Sequencing data revealed that between 3 and 13% of the yeast genome was recovered from each cell, a recovery rate comparable to current single-cell sequencing technologies (FIG. 4C-4D). Importantly, there was minimal overlap between the genomic regions recovered from different cells (FIG. 4C), suggesting that this method does not introduce significant bias in amplification and provides a more representative sampling of the genome when analyzing multiple single cells.

The ability to sequence only the newly synthesized DNA from a single cell is a significant advantage of this method. This capability can be particularly valuable for studies investigating transposable element activity, de novo mutation detection, or DNA replication dynamics, where distinguishing newly amplified sequences from unwanted native DNA is essential. Furthermore, by selectively enriching amplified regions, this method prioritizes high-quality genomic data, which may be especially beneficial in resource-limited sequencing applications. However, by washing away non-biotinylated DNA before sequencing, this method significantly reduces contamination, ensuring that only amplified DNA from the target cell is sequenced.

Intracellular Amplification as a Versatile and Robust Platform: The present intracellular amplification platform demonstrates robust performance across diverse experimental conditions, including different species, fixation and permeabilization protocols, and amplification strategies. By validating the method in both eukaryotic and prokaryotic cells, as well as through targeted and genome-wide amplification, its adaptability for a wide range of biological applications is highlighted.

The ability to amplify specific genes or entire genomes in diverse organisms underscores the broad utility of this method. This approach is well-suited for targeted amplification studies focused on specific genomic loci, such as resistance genes in bacteria or barcode sequences in yeast. Genome-wide amplification, in contrast, enables unbiased analysis of genetic variation and de novo genome assembly in both model and non-model organisms.

This flexibility makes intracellular amplification a powerful tool for evolutionary studies, microbial ecology, and translational research. For example, the ability to amplify and sequence 16S regions in bacteria offers a streamlined workflow for microbiome research, while genome-wide amplification in yeast enables high-resolution studies of population genetics and adaptive evolution. Collectively, these results demonstrate that intracellular amplification is a robust and versatile platform capable of addressing a wide range of biological questions across diverse species and experimental conditions.

Comparison with Existing Amplification and Sequencing Techniques: In Situ PCR (IS-PCR) enhances signal for hybridization by utilizing intracellular DNA amplification. This technique has traditionally been used to localize cells containing genetic elements within tissues or tumors. Specific applications of IS-PCR have included detecting HIV, and malaria in human cells, analyzing oncogenes in tumors, and detecting pathogens in environmental samples. However, the widespread adoption of this technique has been stalled by technical issues including, compromised cell membranes, extracellular amplification, non-uniform amplification, and false signals. Further, IS-PCR is usually performed on paraffin-embedded samples which harshly restricts downstream applications such as sorting labeled cells for ‘omics analyses of genotypes of interest.

When comparing the present method to other amplification and intracellular techniques, several key advantages become apparent. Unlike the more common single-cell RNA-sequencing approaches, which are restricted to transcriptomic analysis, the present method enables the amplification and subsequent sequencing of genomic DNA from individual cells, allowing for the detection of rare mutations, structural variations, and large genomic regions that may be missed in transcriptomic studies. Whole genome amplification (WGA) techniques vary in their efficiency and bias, yet this approach, utilizing random hexamers, ensures an unbiased and comprehensive view of a cell's genetic makeup. This stands in contrast to methods that are restricted to gene-specific primers or are prone to coverage gaps and amplification biases. By leveraging intracellular amplification, this method improves genomic coverage and variant detection, addressing challenges commonly faced in single-cell sequencing.

Another advantage of this approach over traditional amplification techniques, is its ability to work with fixed cells. Conventional multiple displacement amplification (MDA) and other whole-genome amplification methods typically require the lysis of live cells, which can present challenges in applications where cell viability is difficult to maintain or where long-term sample preservation is necessary. This limitation has prompted the development of alternative methods with improved genome coverage and reduced amplification bias, though many still struggle with fixed samples. By enabling whole-genome amplification from fixed cells, this method overcomes these constraints and expands the scope of potential applications, particularly in clinical and environmental settings where sample integrity and preservation are essential.

Implications: This method presents a significant step forward in both single-cell and pooled sequencing, addressing long-standing challenges in the field. Traditional single-cell sequencing is constrained by issues of low yield and contamination. The ability to obtain high-quality sequencing data from amplified DNA in fixed cells enables deeper exploration of microbial diversity and genome recovery. This advancement could lead to new insights into microbial ecology, where understanding the roles of specific species or genes within complex communities is crucial. For example, microbial ecologists can now track which specific strains contribute to community metabolism and how these strains interact with each other in a given environment, all at the level of individual cells.

Beyond microbial genomics, this technology holds promise for applications in synthetic biology and evolutionary studies. In synthetic biology, it could enable the precise characterization of engineered microbes, facilitating advancements in bioengineering applications. By providing a deeper understanding of microbial genome function and interactions, this approach can help optimize microbial consortia for industrial and environmental applications. In evolutionary biology, this method can help uncover rare adaptive mutations and resolve challenges such as incomplete lineage sorting in evolving populations. This capability allows researchers to gain a more accurate understanding of evolutionary processes by directly tracking genomic changes over time and across lineages.

Furthermore, the ability to sequence from fixed cells opens the door to new applications in clinical diagnostics and medical research. Single-cell sequencing technologies have been increasingly recognized for their role in identifying pathogens and drug-resistant strains, even when these are vastly outnumbered by benign microbes in clinical samples. By bypassing the need for isolating individual colonies or conducting extensive enrichment steps, this approach enables direct genomic analysis of microbial populations, facilitating improved disease detection and a deeper understanding of resistance mechanisms. This capability aligns with emerging applications of single-cell sequencing in precision medicine, where rare cell populations, including pathogens, can be accurately identified and monitored for their genomic adaptations.

### Methods:

Intracellular Amplification: One of the central innovations of this method is the ability to amplify genomic DNA directly within fixed and permeabilized cells. By performing amplification intracellularly, biases inherent in traditional single-cell sequencing are mitigated, such as uneven amplification or loss of fragile DNA regions. Instead, this method leverages the cellular environment as a natural reaction vessel.

The selection of an appropriate DNA polymerase was critical for the success of this approach. A thermostable polymerase was employed, optimized for isothermal amplification and capable of functioning despite residual fixative or membrane debris. This polymerase facilitated consistent amplification across various cell types, including yeast and bacteria.

Another key feature of this approach is the intracellular reaction setup. By allowing reagents to diffuse into cells, uniform genome-wide amplification was ensured, avoiding the biases of traditional workflows that rely on DNA extraction, purification, and in vitro amplification. Intracellular amplification preserved genomic integrity and improved the detection of low-abundance sequences, such as rare mutations or transposable elements.

Identifying Highly Amplified Cells: EvaGreen® Staining for Genome Quantification and Sorting: To quantify intracellular genome amplification, cells were (in this embodiment) stained with EvaGreen®, a DNA-binding dye that fluoresces upon binding to double-stranded DNA. Cells were incubated with EvaGreen® for 30 minutes to ensure sufficient dye uptake while minimizing background fluorescence. Following incubation, excess dye was removed by centrifugation, and the cells were washed to prevent non-specific staining.

Fluorescence intensity was measured using an Attune flow cytometer, allowing for precise quantification of amplified DNA within individual cells. Cells exhibiting high fluorescence-indicative of robust genome amplification-were identified and selected for sorting in this embodiment. Using fluorescence-activated cell sorting (FACS), highly amplified cells were isolated and deposited into individual wells of a 96-well plate for downstream processing. This approach enabled the targeted recovery of cells with successful intracellular amplification, improving the efficiency and accuracy of genomic analysis.

DNA Recovery and Sequencing Preparation: Capturing and Amplifying Biotinylated Genomes: Following intracellular amplification, cells were lysed using proteinase K to release genomic material. To inactivate the proteinase K, 5 μL of 100 μM PMSF (resuspended in isopropanol) was added to each tube immediately after heat treatment, followed by a 10-minute incubation at room temperature.

For selective capture of biotinylated DNA in this embodiment, 100 μL of resuspended streptavidin-coated C1 magnetic beads were added to each sample and incubated at room temperature for 60 minutes with agitation. The samples were then placed on a magnetic rack, and the supernatant was removed. Beads were resuspended in 250 μL of 1×wash solution, agitated for 5 minutes, and subjected to two consecutive wash steps. After the final wash, beads were resuspended in 250 μL of 10 mM Tris-HCl (pH 8.0) with 0.1% Tween-20. At this stage, beads could either be rinsed with molecular-grade water and processed immediately or stored overnight at 4° C. in a Tris-Tween buffer.

Captured DNA was re-amplified using phi29 polymerase with random hexamers to generate sufficient material for sequencing. The amplified DNA was then, in this embodiment, prepared for Illumina sequencing, ensuring high-quality genomic data for downstream analysis.

Yeast cell culture. S. cerevisiae C5W4 WTC-SOD1-D102S was used. Cells were streaked on YP plus 2% dextrose agar plates from frozen −80° C. glycerol stocks and grown at 30° C. for 48 hours. Single colonies of (yeast strain) were inoculated into YP plus 2% dextrose liquid media (YPD) and grown at 30° C. with shaking [200 rotations per minute (rpm)] for 24 hours.

Bacterial cell culture. E. coli DH5a was used. Cells were streaked on LB agar plates from frozen −80° C. glycerol stocks and grown at 37° C. for 24 hours. Single colonies of DH5a were inoculated into fresh LB medium and grown at 37° C. with shaking [200 rotations per minute (rpm)] for 24 hours.

Cell Lysis. After amplification, the entire reaction volume was transferred into a microcentrifuge tube and 1 mL of cold PBS and 5 μL of 10% Triton X-100 was added. Samples were spun down at 4° C., 5000 g for 3 min. The supernatant was carefully aspirated off, leaving ~30 μL to avoid removing the pellet. Cells were then resuspended in 1 mL cold PBS, spun down, and resuspended in 1 mL cold PBS for a total of two washes. After washing cells, the supernatant was aspirated off and cells were resuspended in 50 μL of cold PBS, 50 μL of 2×lysis buffer [20 mM Tris (pH 8.0), 400 mM NaCl, 100 mM EDTA (pH 8.0), and 4.4% SDS], and 10 μL of proteinase K solution (20 mg/mL). Cells were incubated at 55° C. for 2 hours with periodic vortexing to lyse the cells and reverse the formaldehyde cross-links.

Preparing Streptavidin Beads for Sample Binding. To prepare beads for sample binding, they must first be washed. For each lysate, 44 μL of Dynabeads MyOne Streptavidin C1 (Invitrogen) were washed three times with 800 μL of a 1×wash solution of 5 mM Tris-HCl pH 8.0, 1 M NaCl, 500 UM EDTA, and 0.05% Tween-20 using a magnetic 1.5 mL tube rack. The beads were then resuspended in 100 μL per sample of a 2×wash solution containing 10 mM Tris-HCl PH 8.0, 2 M NaCl, and 1 mM EDTA.

Sample Binding to Streptavidin Beads. Directly after lysates were removed from heat, 5 μL of 100 UM PMSF (resuspended in isopropanol) was added to each tube and incubated at room temperature for 10 minutes to inactivate the proteinase K. To bind DNA to C1 beads, 100 μL of resuspended C1 beads were added to each sample tube and agitated at room temperature for 60 minutes. The samples were then placed on the magnetic rack and the supernatant was removed. Samples were removed from the magnetic rack and resuspended in 250 μL of the 1×wash solution and agitated at room temperature for 5 min. Samples were replaced on the magnetic rack and the subsequent steps were repeated for a total of two wash steps. After the two 1×wash steps, the supernatant was removed, and the samples were resuspended in 250 μL of 10 mM Tris-HCl pH 8.0 and 0.1% Tween-20. At this point, the beads could be rinsed with 250 μl of molecular-grade water while the beads were still bound to the magnetic rack and moved on to subsequent steps or resuspended in 250 μL of the Tris-HCL Tween-20 buffer and stored at 4° C. overnight.

Off-the-beads Amplification. If the samples were stored overnight in the Tris-Tween buffer, the tubes were placed in a magnetic rack and the beads were rinsed with 250 μL of molecular-grade water. The bead-bound DNA was then amplified in 220 μL reactions with 2×high fidelity polymerase (KAPA HiFi) and 0.4 μM BC_0062 and BC_0108 primers for 3 min at 95° C., and then five cycles of 98° C. for 20 s, 65° C. for 45 s, and 72 for 3 min.

Quantitative PCR. The off-the-beads PCR product was then placed against a magnetic rack and the supernatant was transferred to new optical-grade PCR tubes with qPCR dye (EvaGreen® 20×). The samples were then amplified on a qPCR machine for a further 10-20 cycles until the amplification curves exited the log-linear phase.

Size selection bead clean up. The PCR products were then cleaned using a 0.8×SPRI size selection and eluted in 20 μL of molecular-grade water.

Gel electrophoresis. 5 μL of the product which was eluted during the bead clean-up was then run on a 2% agarose gel at 120 V for 15-20 min. A single properly amplified gene should appear as a dark band on the gel at the correct size of the gene. The genome that has been properly amplified with random hexamers should appear as a smear starting at approximately 5-7 KB and ending at approximately 300 bp on a gel.

### Example 2

The following are non-limiting examples of fixation and permeabilization described by the present invention. It is to be understood that said example is not intended to limit the present invention in any way. Equivalents or substitutes are within the scope of the present invention.

Fixation and permeabilization is an important step in some of the methods of the present invention, as it preserves cellular integrity while rendering cells permeable to the amplification reagents. This balance is essential for intracellular genome amplification, where cellular structures must remain intact to contain the reaction while allowing for efficient diffusion of reagents. To this end, a range of fixation methods were explored to identify conditions that optimize these competing needs.

Formaldehyde-based fixation emerged as a versatile approach. Formaldehyde crosslinks proteins, stabilizing cellular architecture while preserving nucleic acids. Multiple protocols were tested, described below, including room-temperature fixation for short durations and overnight fixation at 4° C., a method for preserving yeast cells. Both approaches demonstrated consistent results in maintaining cell morphology and enabling downstream amplification.

Ethanol fixation/permeabilization, another commonly used method, offered a complementary approach. Ethanol rapidly precipitates cellular proteins and lipids, creating a highly permeable cellular state. While less gentle than formaldehyde, ethanol fixation proved effective in permeabilizing bacterial cells such as E. coli DH5α for this workflow, described below. This method is particularly advantageous for its simplicity and speed, as cells can be processed in under 15 minutes.

Interestingly, the choice of fixation method did not significantly impact the efficiency of genomic amplification or fluorescent labeling, highlighting the robustness of this technique. However, subtle differences in cell robustness and fluorescence intensity were noted. Without wishing to limit the present invention to any theory or mechanism, this may suggest that FISH fixation may better preserve intracellular targets for flow cytometry applications.

Following fixation, permeabilization was required to facilitate the entry of amplification reagents. A variety of methods were tested, including enzymatic treatments with zymolyase for yeast cell walls and detergents such as Tween-20 and Triton X-100 for broader applicability. Each approach was tailored to the unique properties of the cell type and experimental needs. For yeast, zymolyase digestion of the cell wall was particularly effective, transforming the rigid cell into a spheroplast while maintaining structural integrity. In addition, detergent-based methods provided a rapid and scalable alternative for both yeast and bacterial cells.

These optimizations of fixation and permeabilization enabled intracellular amplification across diverse cell types. This approach refines existing protocols to meet the specific demands of (in some embodiments) fluorescently labeled intracellular genome amplification.

Below are non-limiting examples of methods that may be used for fixation and permeabilization.

Overnight Formaldehyde Fixation with Zymolyase and Tween-20 Permeabilization: At the time of sampling, 3 mL of yeast cultures, grown to saturation in YPD medium, were immediately spun down in a room-temperature centrifuge at 5000 g for 3 minutes. The cell pellet was fixed with 4% formaldehyde and incubated overnight at +4° C. on a shaker. Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. Permeabilization was performed with 0.1% Tween-20 at room temperature for 10 minutes before proceeding to downstream steps.

Overnight Formaldehyde Fixation with Zymolyase and TritonX-100 Permeabilization: At the time of sampling, 3 mL of yeast cultures, grown to saturation in YPD medium, were immediately spun down in a room-temperature centrifuge at 5000 g for 3 minutes. The cell pellet was fixed with 4% formaldehyde and incubated overnight at +4° C. on a shaker. Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. Permeabilization was performed with 0.1% TritonX-100 at room temperature for 10 minutes before proceeding to downstream steps.

10 Minute Formaldehyde Fixation with Zymolyase and TritonX-100 Permeabilization (“FISA fix”): At the time of sampling, 3 mL of yeast cultures, grown to saturation in YPD medium, were immediately spun down in a room-temperature centrifuge at 5000 g for 3 minutes. The cell pellet was fixed with 4% formaldehyde for 10 minutes at room temperature (+20° C.). Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. Permeabilization was performed with 0.1% TritonX-100 at room temperature for 10 minutes before proceeding to downstream steps.

30 Minute Formaldehyde Fixation with Zymolyase and Overnight Ethanol

Permeabilization (“FISH fix”): At the time of sampling, 3 mL of yeast cultures, grown to saturation in YPD medium, were immediately spun down in a room-temperature centrifuge at 5000 g for 3 minutes. The cell pellet was fixed with 4% formaldehyde for 30 minutes at room temperature (+20° C.) and then transferred to +4° C. for overnight fixation on a shaker. Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. The permeabilization step used ethanol treatment overnight at room temperature before downstream steps.

Ethanol Fixation and Permeabilization: At the time of sampling, 3 mL of Escherichia coli cultures, grown to the desired density, were immediately spun down in a room-temperature centrifuge at 5000 g for 3 minutes. The cell pellet was resuspended in 70% ethanol and incubated at room temperature (+20° C.) for 10 minutes. Fixed cells were centrifuged again at 5000 g for 3 minutes, the supernatant was discarded, and the pellet was resuspended in ice-cold PBS. This washing step was repeated twice to ensure the removal of excess ethanol. Fixed cells were then processed directly for intracellular amplification or other downstream applications.

### Example 3

The following is a non-limiting example of additional methods that may be used in the methods described in the present invention. It is to be understood that said example is not intended to limit the present invention in any way. Equivalents or substitutes are within the scope of the present invention.

Genomic DNA Amplification with EdU Labeling. To prepare the amplification reaction, cells were resuspended in the phi29 reaction mix, which contained 10×phi29 buffer, BSA (20 mg/mL), dNTPs (10 mM), sorbitol, phi29 polymerase (10,000 U/mL), primers (25 μM), and EdU (10 μM) as per Table 1. Reactions were incubated at 30° C. for 16 hours, and EdU incorporation proceeded throughout the amplification.

TABLE 1
phi29 Reaction Mix Composition

| Reagent | Concentration | | Amount per reaction | |
|---|---|---|---|---|
| 10X phi29 buffer | 10X | | 2.5 | μL |
| BSA | 20 | mg/mL | 0.25 | μL |
| dNTPs | 10 | mM | 1.5 | μL |
| EdU | 10 | μM | 1 | μL |
| Sorbitol | 27%, 1.8M, 2M | | 5.75 | μL |
| phi29 polymerase | 10,000 | U/mL | 1 | μL |
| Primers | 25 | μM | 8 | μL |
| Cells | 1 | million/mL | 5 | μL |

EdU Detection. Following amplification, cells were washed twice with 3% BSA in PBS to remove excess phi29 reaction buffer. The Click-iT® reaction cocktail was prepared fresh according to Table 2 and added to each sample. Cells were incubated at room temperature in the dark for 30 minutes, washed with 3% BSA in PBS, and prepared for imaging or further analysis.

TABLE 2
Click-iT ® Reaction Cocktail Composition.

| Component | Volume (per reaction) | |
|---|---|---|
| 1X Click-iT ® reaction buffer | 430 | μL |
| CuSO₄ (Component E) | 20 | μL |
| Alexa Fluor ® azide | 1.2 | μL |
| Reaction buffer additive | 50 | μL |

The cocktail was used within 15 minutes of preparation to ensure reaction efficiency.

Flow Cytometry Analysis of EdU-Labeled Cells. After the Click-iT® EdU detection step, cells were washed twice with 1 mL of 3% BSA in PBS to remove excess reagents. The final cell pellet was resuspended in 200 μL of cold PBS and kept on ice until analysis. For flow cytometry, 100 μL of the cell suspension was loaded into an Attune NXT Flow Cytometer (Thermo Fisher Scientific). The instrument was calibrated prior to each experiment to ensure consistent performance. Fluorescence signals from Alexa Fluor®-tagged EdU-labeled DNA were measured using the appropriate laser and filter settings (e.g., excitation at 488 nm, emission at 530/30 nm for Alexa Fluor® 488). At least 10,000 events were recorded per sample. Data were analyzed using Attune NXT software, with gating strategies applied to exclude debris and doublets and to focus on single-cell populations. Results were reported as fluorescence intensity histograms and analyzed to determine the extent of EdU incorporation in amplified DNA.

### Example 4

The following is a non-limiting example of the possible applications of the methods described in the present invention. It is to be understood that said example is not intended to limit the present invention in any way. Equivalents or substitutes are within the scope of the present invention.

Targeted Amplification with Specific Primers vs. Genome-Wide Amplification with Random Hexamers: By selecting specific primers, this method enables targeted amplification of genomic loci of interest, such as genes associated with particular phenotypes or adaptive mutations. For example, using primers designed to amplify drug-resistance genes in a microbial community may be used to help identify and isolate cells contributing to multidrug resistance. This targeted approach contrasts with genome-wide amplification using random hexamers, which is ideal for unbiased single-cell sequencing or the detection of novel mutations across the entire genome. Unlike existing methods that often require multiple separate experiments for targeted and genome-wide approaches, the present platform integrates both into a single framework, reducing time and resource expenditure.

Sorting Strategies: Pooling vs. Single-Cell Sorting: This method's compatibility with different sorting strategies allows researchers to adapt the workflow to the resolution required for their study. Pooling fluorescently labeled cells and sequencing them together is an efficient strategy for population-level studies, such as tracking the prevalence of specific genotypes in an evolving microbial community. In contrast, single-cell sorting provides the granularity needed to explore cellular heterogeneity, identify rare subpopulations, or reconstruct lineage-specific genomic changes. For instance, single-cell sorting would be essential for studies aiming to resolve clonal populations within tumors or trace the genetic basis of antibiotic resistance within a bacterial population.

Selective Sequencing of Amplified DNA Using Biotinylated Primers: By incorporating biotinylated primers into the amplification process in this embodiment, this method can selectively enrich for newly synthesized DNA. This feature is particularly advantageous when pre-existing genomic DNA or background contamination needs to be excluded from downstream analysis. For example, biotinylated primers can be used to isolate and sequence only newly replicated DNA during studies of DNA replication dynamics or to investigate transposable element activity without interference from the original genomic template.

Applications and Customizable Workflows: Potential workflows and their corresponding applications include: a) Targeted amplification with pooled sorting for characterizing population-level diversity or detecting the presence of specific genotypes in environmental samples or microbial communities, b) Random hexamer amplification with single cell sorting for investigating rare mutations within a complex population, such as single-nucleotide polymorphisms (SNPs) or structural variations, c) Biotinylated primer-based amplification is ideal for some studies, d) A combination of FISA and genome-wide amplification can provide organism context alongside genetic information, enabling studies of mixed microbial populations where some if not all individuals are unknown or don't have a reference genome

These customizable workflows address the limitations of current methods by integrating precision, scalability, and versatility into a single platform. For example, unlike traditional fluorescence in situ hybridization (FISH), which is limited to visualizing specific loci, this method enables subsequent sequencing of the amplified DNA for detailed genetic analysis. Similarly, existing single-cell sequencing approaches often require extensive pre-amplification steps that can introduce biases or exclude low-abundance templates, challenges that this platform overcomes by combining efficient intracellular amplification with targeted or genome-wide approaches.

Overall, the versatility of this method not only broadens its applicability across diverse biological fields, but also offers researchers the freedom to design workflows that align with their unique experimental goals. This adaptability positions the present platform as a powerful tool for advancing genomic research in evolutionary biology, medicine, and microbial ecology.

### Example 5

The following is a non-limiting example of the possible applications of the methods described in the present invention. It is to be understood that said example is not intended to limit the present invention in any way. Equivalents or substitutes are within the scope of the present invention.

Here, intracellular genomic amplification (INgen) is introduced. INgen is a method that harnesses the cell membrane as a natural reaction chamber to amplify DNA within fixed, permeabilized cells. INgen employs a strand-displacing, isothermal polymerase (optionally) together with biotinylated primers and/or (optionally) together with fluorescent techniques, to achieve robust intracellular DNA amplification and efficient recovery of the amplified material for sequencing. This approach overcomes a long-standing barrier that has prevented important advances in single-cell and rare-cell genomics: the inability to amplify and recover sequenceable DNA from fixed cells. By pushing past this barrier, INgen provides a critical and previously inaccessible step toward scalable single-cell DNA sequencing without the need to isolate each cell into a separate reaction vessel. Using INgen, targeted and whole-genome amplification is demonstrated across diverse organisms, including Saccharomyces cerevisiae, Bacillus subtilis, and Escherichia coli. Together, these capabilities position INgen as a foundational advance that paves the way for the next generation of single cell sequencing methods including high-throughput single-cell sequencing without physical isolation, contamination-resistant amplification within intact cells, and rare-cell enrichment prior to genomic analysis.

Single-cell DNA sequencing has the potential to transform the ability to study genetic heterogeneity in mixed microbial communities (e.g., microbiomes) and in any population where cells are genetically diverse (e.g., tumors, drug-resistant infections, laboratory experiments, etc.). However, a major bottleneck for many single-cell ‘omics technologies is the dependence on physical cell isolation, which limits scalability and throughput. As a result, single-cell genomics studies are often limited to just hundreds or even dozens of cells. Additionally, isolation techniques often demand time-intensive labor, specialized expertise, and expensive instrumentation, further limiting their accessibility. Ironically, each cell's membrane already naturally compartmentalizes its genetic material, providing a built-in reaction chamber. Yet, sequencing reactions are still carried out in separate vessels, a redundant and inefficient step given that the cell itself is perfectly isolated. This inherent feature suggests that using the cell as its own reaction chamber can significantly streamline single-cell genomic workflows.

Intracellular DNA-based applications have not been widely implemented due to several technical challenges, including the hurdle of amplifying DNA in formaldehyde-fixed cells in such a way that allows it to be sequenced. A robust method for intracellular DNA amplification that allows for downstream DNA sequencing thus stands to enable the optimization of higher-throughput single-cell DNA sequencing protocols.

A significant challenge in intracellular genomics arises from the need to fix cells with formaldehyde in order to turn them into sturdy reaction chambers. Fixing with formaldehyde stabilizes the cell and the genetic material inside, but the crosslinks that are generated are frequently cited as obstacles to efficient DNA amplification and can hinder sequencing. Sequencing of genomic DNA that was amplified inside of formaldehyde-fixed cells has heretofore not been possible. Instead, recent intracellular methods randomly insert transposons that drive transcription of DNA to RNA, avoiding the challenge of intracellularly amplifying and sequencing DNA.

To address this and other challenges, intracellular genomics (INgen) is hereby introduced. INgen is a novel method that enables intracellular DNA amplification and subsequent DNA sequencing. By overcoming long-standing barriers, this advance has the potential to unlock important (optional) downstream applications, such as higher throughput single-cell DNA sequencing without the need for cell isolation. The methodological details of INgen are reported herein, including the key innovations that explain why it works. Also reported herein is successful sequencing of target genes up to 3 kb and whole genomes in diverse organisms, including S. cerevisiae, B. subtilis, and E. coli. Control experiments are also reported herein, confirming that the sequenced DNA originates from intracellular amplification, rather than leaked or native genomic DNA. It is also shown that up to 14% of the yeast genome can be amplified within a single cell. Finally, successful (optional) fluorescence-activated cell sorting and sequencing of cells containing intracellularly amplified DNA is demonstrated.

In sum, INgen is a robust and versatile method for intracellular DNA amplification in diverse cell types, paving the way for a wide array of (optional) downstream applications, including high-throughput single-cell sequencing, genotype-based cell sorting, and rare cell genomics. By breaking this critical barrier of amplifying and sequencing DNA from fixed cells, INgen stands to enable advances in single-cell ‘omics with broad potential applications in microbial ecology, cancer genomics, and evolutionary biology.

A flexible method for intracellular DNA Amplification (INgen): The challenge of amplifying DNA within cells and sequencing this DNA is addressed herein. One challenge associated with intracellular DNA amplification is that the temperature cycling required for polymerase chain reaction can weaken cell membranes and cause DNA leakage. Another challenge is that the formaldehyde cross-links that turn fixed cells into sturdy reaction chambers come with a negative side effect: they block enzymes such as DNA polymerase from accessing DNA. Use of Phi29 polymerase (or other isothermal polymerases) contributes to the success described herein in overcoming these challenges. Phi29 polymerase (or other isothermal polymerases) can overcome formaldehyde crosslinks through their robust strand-displacement activity. Furthermore, isothermal polymerases (including but not limited to Phi29) do not require the thermal cycling that has been associated with membrane disruption, increased risk of extracellular amplification, and false signals. Instead, the protocol described herein includes only a short, controlled pre-incubation at 72° C., prior to polymerase addition, to facilitate primer annealing.

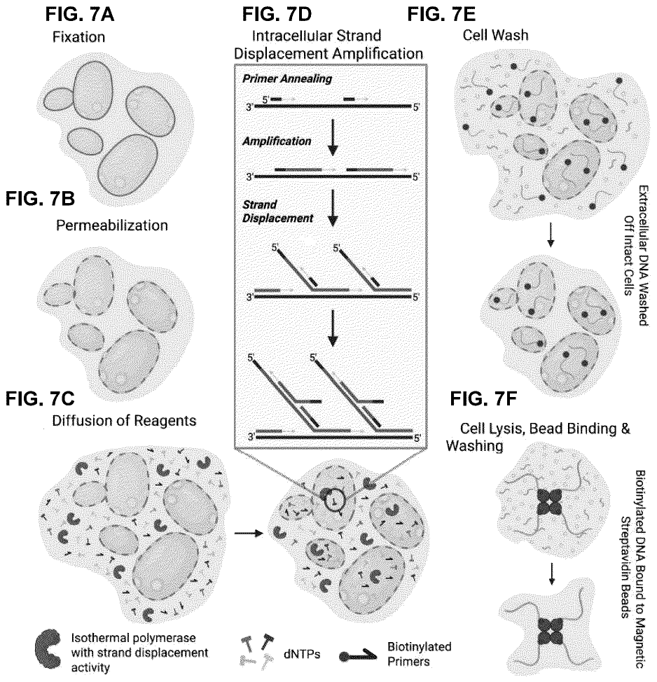

Another way the INgen protocol addresses the challenge of recovering DNA from formaldehyde-fixed cells is through (optional) biotinylated primers. These optionally enable the selective capture of intracellularly amplified DNA on magnetic streptavidin beads following cell lysis. Streptavidin-bound DNA can be washed multiple times, allowing removal of harsh reagents used during the lysis of fixed cells. This purification step helps ensure that subsequent amplification reactions, such as those used for preparing DNA for sequencing, can proceed efficiently. The INgen workflow consists of four key steps: (1) fixation and permeabilization of cells to stabilize the cellular structure and allow reagent access (FIG. 7A-7C), (2) intracellular DNA amplification using an isothermal polymerase (FIG. 7D) and (optionally) biotinylated primers, (3) cell washing, cell lysis, and (optionally) isolation of newly synthesized DNA via streptavidin-coated beads (FIG. 7E-7F), and (4) optional downstream sequencing of amplified genomic material. It was demonstrated that intracellular amplification and subsequent sequencing of the amplified products is not only feasible but also effective across multiple contexts, underscoring the utility of this method for downstream single-cell sequencing applications.

To assess the applicability of the INgen method across taxa, intracellular amplification was tested in the eukaryotic model organism Saccharomyces cerevisiae (yeast) and two prokaryotic species, Bacillus subtilis (a gram-positive bacterium) and Escherichia coli (a gram-negative bacterium). Using species-specific fixation and permeabilization protocols, genomic material was successfully amplified in all organisms. For yeast, fixation with formaldehyde followed by treatment with zymolyase and Triton-X enabled permeabilization, while ethanol fixation alone was sufficient for E. coli and B. subtilis, and did not require additional permeabilization steps. These results demonstrate that the INgen platform is robust to the distinct cell wall and membrane compositions of Gram-negative and Gram-positive bacteria, as well as fungi, making it broadly applicable across phylogenetically diverse organisms.

Intracellular amplification of targeted regions and recovery of DNA for sequencing: To evaluate the ability of INgen to selectively amplify specific genomic regions, (optional) biotinylated primers were designed, targeting genes of varying lengths, including three genes from yeast (SOD1, 450 bp; PDR3, 3,000 bp; and an engineered barcode BC, 300 bp) and one gene from bacteria (16S rRNA, 1,000 bp). For each reaction, the INgen protocol was performed on roughly 1 million cells of the target cell type following the method described above (see also FIG. 7A-7F). Optional biotinylated primers were annealed to DNA during a 5-minute incubation at 72° C., performed after all reaction components, except Phi29 polymerase, had been diffused into the cells. Following this step, Phi29 polymerase was added, and cells were incubated for 16 hours at 30° C. After incubation, cells were lysed, and the newly synthesized, biotin-labeled DNA was (optionally) isolated using streptavidin-coated beads. Once bound and washed, a secondary ‘off-the-beads’ amplification reaction using targeted primers was performed to generate sufficient DNA for library preparation, which prepares DNA for optional next-generation sequencing.

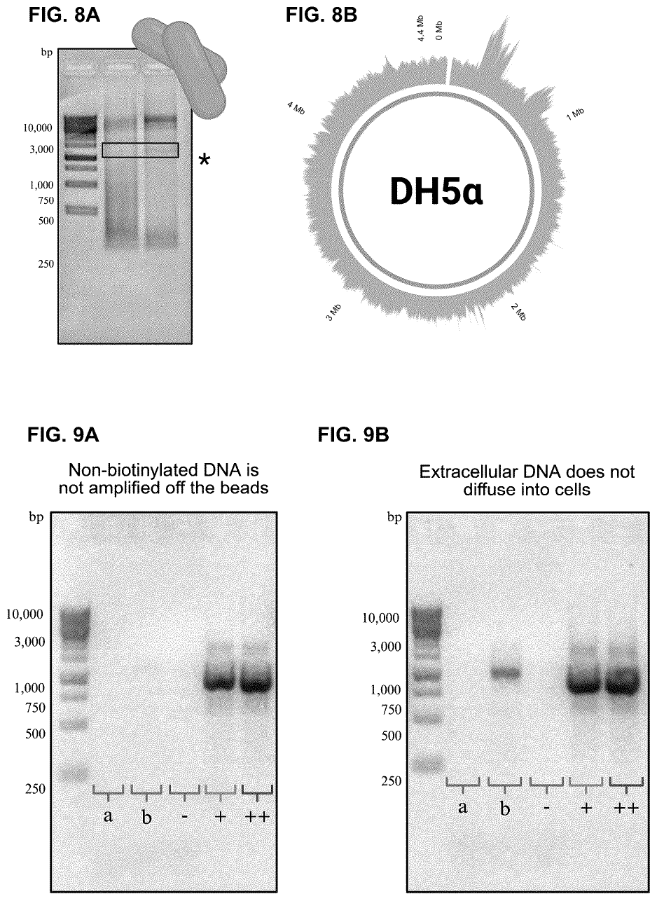

Gel electrophoresis analysis confirmed successful targeted amplification off-the-beads, with distinct bands of the expected sizes observed for all tested loci (FIG. 8A). While amplification of any regions above 3 kb was not attempted, the highly processive nature of phi29 and whole genome data collected herein suggests that longer targets can also be amplified. Following size confirmation on an agarose gel, DNA fragments were prepared for (optional) sequencing using the Illumina DNA Flex kit. Targeted genomic regions were (optionally) successfully sequenced to a minimum depth of 15× (FIG. 8B). The ability to target multiple genes across multiple species highlights the flexibility and broad applicability of this method.

To confirm that DNA amplification is happening intracellularly, rather than extracellularly on leaked DNA, a control experiment was performed, where varying amounts of bacterial DNA were spiked in a reaction vessel containing yeast cells (FIG. 9A). The INgen protocol was performed on these mixtures using 16S primers targeting the bacterial DNA. After the 16-hour amplification step, the supernatant was removed, and the same 16S primers were used to reamplify 16S bacterial DNA, obtaining clear bands on an agarose gel (FIG. 9A; pre-wash). However, after washing the cells twice, PCR reactions using subsequent supernatants did not produce robust bands (FIG. 9A; wash 2). Given that more DNA was spiked in than would ever be expected to be present extracellularly, and this still could not achieve robust amplification after wash steps, it was concluded that the amplicons produced during a typical INgen reaction (FIG. 8) are not predominantly created extracellularly by priming and elongating DNA fragments that leaked out of cells.

To confirm that the amplicons in FIG. 8 do not reflect amplification of native genomic DNA post-cell lysis, a second control experiment was performed (FIG. 9B). Native genomic DNA should be washed away post-cell lysis, leaving only streptavidin-bound products of intracellular amplification. To test this, large amounts of non-bead-bound bacterial 16S DNA were spiked into reactions post-cell-lysis. Amplification reactions performed using the supernatant from subsequent wash steps failed to produce robust 16S products on an agarose gel after just two washes (FIG. 9B). A control experiment was also performed to confirm that non-bead-bound DNA cannot associate with streptavidin beads (FIG. 9). These control experiments support the conclusion that amplification is specific to intracellularly amplified, biotinylated DNA and not native genomic DNA that fails to be washed away. (Also see FIG. 4, where intracellular amplification was confirmed by showing cells that underwent INgen have a stronger fluorescent signal after DNA stain).

While intracellular DNA amplification has been attempted before, prior methods produced extremely limited (to the point of that amounts produced were essentially negligible, and unsuitable for subsequent downstream steps, including but in no way limited to sequencing) or unusable material that could otherwise not be sequenced. INgen overcomes this barrier by enabling robust intracellular amplification and recovery of sequenceable DNA across diverse cell types and genomic targets. Multiple controls confirm that amplification occurs within cells rather than from leaked or native DNA. By making it possible to sequence DNA amplified inside fixed cells, INgen has the potential to transform what was previously a technical dead end into a versatile platform for single-cell genomics.

Whole genome amplification with random hexamers: Beyond targeted genes, it was next examined whether INgen could be extended to amplify and sequence entire genomes. To test this, INgen was performed using biotinylated random hexamers in ~1 million E. coli cells. Following intracellular amplification, the newly synthesized, biotin-labeled DNA was isolated, re-amplified off the beads using random hexamers, and the resulting DNA pool was sequenced.

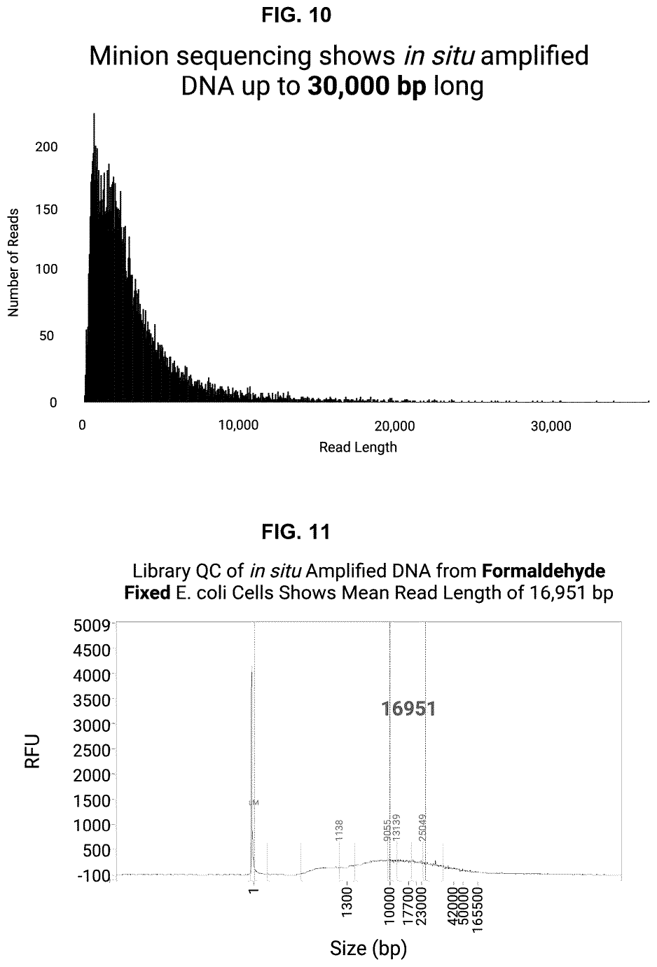

Gel electrophoresis of the off-the-beads PCR product revealed smears spanning a broad range of fragment sizes, consistent with successful whole-genome amplification. Fragment sizes ranged from 200 bp to 50 kb (FIG. 8A). TapeStation analysis further confirmed that intracellular amplification generated long DNA fragments, with an average size of ~20 KB, and some spanning up to several hundred kilobases (FIGS. 10, 11, & 12), consistent with the expected performance of phi29 polymerase. These results suggest that formaldehyde crosslinking does not significantly inhibit this polymerase's activity in the described reaction conditions.

The collective reads from many INgen-amplified cells reconstructed the entire E. coli genome (FIG. 8B). From a single intracellular PCR reaction containing approximately one million cells, 99.4% of the E. coli genome was covered at a depth greater than 30× (FIG. 8B). These results demonstrate that INgen supports robust amplification across diverse genomic loci within fixed cells, expanding its utility beyond targeted assays and setting the stage for comprehensive, isolation-free, single-cell genome sequencing, provided that each cell's DNA could be barcoded in a similar way as is done for RNA.

The INgen method can also be deployed to address a persistent challenge in traditional isolation-based single-cell whole-genome sequencing: contamination. Because isolation-based scDNAseq experiments begin with extremely limited DNA (that from a single isolated cell), even trace amounts of exogenous DNA can be amplified along with the genome of interest. Random hexamers exacerbate this problem since they bind indiscriminately to any DNA, including contaminants introduced from reagents, tubes, or gloves. INgen mitigates this issue by using the cell itself as a sealed reaction vessel, thereby physically restricting amplification to the DNA already present within that cell. In addition, the use of biotinylated primers enables selective recovery of intracellularly amplified DNA, while wash steps remove non-biotinylated or extracellularly biotinylated DNA (FIGS. 6 & 9). Together, these features can minimize contamination and improve the accuracy and reliability of single-cell genomic sequencing.

Successful sorting and sequencing of individual cells with intracellularly amplified DNA: To further validate that DNA amplification is occurring intracellularly, control experiments were designed in yeast cells, where either polymerase or primers were omitted from the intracellular reactions. Control and test yeast cells were then stained with EvaGreen®, a fluorescent dye that binds to all double-stranded DNA, and fluorescence distributions were compared. Indeed, test populations exhibited significantly greater average fluorescence compared to controls because the test reactions possessed all necessary reagents to intracellularly amplify DNA (FIG. 4A). This provides further evidence that amplification is occurring intracellularly.

Next, flow cytometry was used to sort and select test cells exhibiting brighter fluorescence than measured in any control cell (FIG. 4A). The fluorescent cells that met the selected criteria were sorted into individual vessels so that it could be confirmed that they each contained intracellularly amplified DNA that is viable for sequencing. The primary objective was to determine if the brightest cells indeed contained sequenceable, biotinylated DNA. The secondary objective was to learn what fraction of the genome was amplified within the brightest cells.

4 single cells were processed. Each cell was lysed using Proteinase K, and the newly synthesized, biotinylated DNA was captured with streptavidin-coated beads and washed several times to remove native genomic DNA (FIG. 4B). For all four cells, the bead-bound DNA was then re-amplified using random hexamers, which generated enough starting material (>5 ng/uL) to construct sequencing libraries following optional sequencing. This suggests all four cells contained biotinylated DNA amplicons. Next, it was asked how much of the genome had been amplified in any one cell. Reported single-cell whole-genome recovery yields can sometimes be as high as ~80-90% in some mammalian systems. However, in challenging contexts, such as microbial or fixed cells, recovery rates can be an order of magnitude lower. In such systems, researchers often reconstruct complete genomes computationally by clustering partial genome fragments across multiple cells or bins.

In terms of assembling genomes by clustering partial reads from many cells, a key strength of INgen is that intracellular amplification can be performed in parallel across thousands of pooled cells. INgen may be optionally paired with combinatorial indexing approaches. Such coupling can enable genome recovery by clustering partial, cell-labeled genomes, potentially enhanced by primers that target anchor regions such as 16S rRNA genes. Even a few percent of a single cell's genome may therefore be sufficient for this kind of analysis, as demonstrated in low-coverage single-cell RNA-seq studies where sparse transcriptome coverage still resolves cell-type relationships.

Because DNA recovery from formaldehyde-fixed cells has historically been so difficult, the first aim was to test whether enough amplified DNA could be obtained to make any (optional) downstream analyses conceivable. Given that the cells with the strongest fluorescent signal were selected from, this experiment will not report the average amount of intracellular amplification per cell, but the upper limit, given the extent of optimization thus far. Sequencing data revealed that between 3 and 13% of the yeast genome was recovered from each of the four cells that were processed. There was minimal overlap between the genomic regions recovered from different cells (FIG. 4C), such that combining reads from all four cells resulted in the recovery of 29.44% of the yeast genome. This high level of per-cell coverage demonstrates that INgen is capable of generating sufficient information for accurate genome reconstruction via cell clustering and thus provides a path toward high-throughput single-cell DNA sequencing.

In sum, INgen establishes a versatile foundation for next-generation single-cell genomics. By enabling optional downstream sequencing of DNA from formaldehyde-fixed cells after (in some embodiments) FACS, it can transform rare-cell genomics, allowing researchers to sort desired genotypes based on (optionally) fluorescence (and/or other optional sorting techniques, e.g., biotinylation) and then sequence the enriched genotypes within the sorted pool. INgen can also complement existing isolation-based scDNAseq approaches by pre-amplifying intracellular DNA to boost signal and reduce contamination. Most excitingly, INgen opens the door to (optional) high-throughput single-cell DNA sequencing without physical isolation, since combinatorial barcoding strategies already proven for RNA can now be applied to DNA. By overcoming the key barrier to amplifying and sequencing DNA inside fixed cells, INgen sets the stage for scalable, pooled single-cell genomics across diverse biological systems.

INgen breaks a decades-old barrier: it enables robust amplification and (optional) recovery of DNA from within fixed cells. This is a prerequisite step that has long prevented scalable single-cell DNA sequencing. INgen overcomes major challenges that previously made intracellular DNA amplification and (optional) sequencing impossible. Using phi29 polymerase, a highly processive, strand-displacing enzyme, strong intracellular amplification was achieved under isothermal conditions, avoiding the high-temperature cycles that damage membranes. Incorporating (optional) biotinylated primers allowed selective capture of newly synthesized DNA after lysis, enabling (optional) downstream purification and sequencing. Across all tested conditions, different fixation and permeabilization methods, species (yeast, E. coli, and Bacillus subtilis), and amplification targets, INgen performed robustly. Targeted amplification and (optional) sequencing of specific genomic loci was validated, and successful whole-genome amplification in bacteria was demonstrated, recovering over 99% of the E. coli genome from a pooled intracellular reaction. It was further shown that individual yeast cells can yield up to 13% genome recovery, a level sufficient to enable (optional) downstream clustering or reconstruction when applied across many cells. By demonstrating effective intracellular DNA amplification in both prokaryotes and eukaryotes, INgen establishes a generalizable foundation for scalable single-cell DNA analysis. The ability to amplify DNA inside fixed cells without compromising membranes or sequenceability transforms what can now be attempted in single-cell genomics. INgen's design, amplifying DNA intracellularly, (optionally) labeling it via biotinylation and/or fluorescent techniques and/or other appropriate labeling techniques, and then (optionally) purifying it for (optional) sequencing, provides a modular framework for next-generation developments, including (optional) downstream combinatorial barcoding, genotype-based cell sorting, and high-throughput analysis of complex populations such as microbiomes or tumors.

Comparison with Existing Amplification and Sequencing Techniques: INgen is more versatile than other intracellular amplification methods, such as in situ PCR (IS-PCR), because INgen allows (optional, downstream) sequencing of the amplified material. IS-PCR instead has traditionally been used to localize cells containing particular genetic elements within tissues or tumors by enhancing the signal for probe hybridization. Specific applications of IS-PCR have included detecting HIV and malaria in human cells, analyzing oncogenes in tumors, and detecting pathogens in environmental samples. However, the widespread adoption of IS-PCR has been stalled by technical issues, including compromised cell membranes, extracellular amplification, non-uniform amplification, and false signals. Further, IS-PCR is usually performed on paraffin-embedded samples, which restricts downstream applications such as sorting labeled cells for ‘omics analyses of genotypes of interest. INGen overcomes many of the limitations of IS-PCR because it works with cells in solution, amplifies DNA at low temperatures that do not result in compromised membranes, and (optionally) biotinylates the amplicons which eliminates false signals from extracellularly amplified DNA.

INgen also possesses several advantages over conventional multiple-displacement amplification (MDA) methods. MDA requires live-cell isolation and lysis, which limits scalability and complicates work with fragile or fixed samples. INgen overcomes this limitation by performing amplification prior to lysis, allowing DNA to be recovered even from preserved or formaldehyde-fixed cells. This feature expands access to clinical, environmental, and archival samples that cannot easily be processed by existing single-cell techniques. Finally, while single-cell RNA-seq revolutionized transcriptomic analysis, it cannot directly capture genomic variation. INgen complements such methods by enabling intracellular amplification of genomic DNA, paving the way for the (optional, downstream) detection of non-coding mutations, structural variants, and other lineage-defining changes that are invisible to RNA-based assays. In sum, INgen transforms what was once a dead end, the inability to amplify and recover DNA from fixed cells, into a versatile platform for next-generation single-cell genomics. Furthermore, incorporating other variants of phi29 polymerase, or other isothermal polymerases, can further enhance INgen's performance. By overcoming a fundamental barrier, INgen opens new possibilities for scalable, pooled, and barcoded single-cell DNA sequencing across a broad range of biological systems.

Methods: Fixation and Permeabilization: Fixation and permeabilization are crucial steps in INgen, as they maintain cellular integrity while enabling reagent diffusion for intracellular genome amplification. This balance is necessary to preserve cell structures while ensuring effective reagent penetration. To achieve this, multiple fixation strategies were evaluated to identify optimal conditions.

Formaldehyde-based fixation provided a robust approach by crosslinking proteins and stabilizing cellular architecture while preserving nucleic acids. Protocols were tested ranging from short incubations at room temperature (e.g., 10 minutes) to extended fixation at 4° C., a method used for yeast preservation. Both protocols maintained cell morphology and supported efficient downstream amplification.

Ethanol fixation/permeabilization offered an alternative strategy. Ethanol rapidly precipitates proteins and disrupts membranes, enhancing permeability. While harsher than formaldehyde, this method effectively permeabilized bacterial cells such as E. coli DH5a. Its simplicity and rapid processing time (under 15 minutes) made it an attractive option.

Interestingly, the fixation method had minimal impact on genomic amplification efficiency or fluorescent labeling, underscoring the robustness of the INgen approach. However, differences in cell durability and fluorescence intensity were observed, suggesting that formaldehyde fixation may better preserve intracellular targets for flow cytometry.

Following fixation, permeabilization was required to facilitate reagent entry. Enzymatic treatments were tested, such as zymolyase digestion for yeast cell walls, and detergent-based methods using Tween-20 and Triton X-100. Zymolyase treatment efficiently converted yeast cells into spheroplasts while maintaining structural integrity. Detergent-based approaches provided a rapid, scalable alternative applicable to both yeast and bacterial cells.

These optimizations of fixation and permeabilization enabled intracellular amplification across diverse cell types. The INgen approach refines existing protocols to meet the specific demands of fluorescently labeled intracellular genome amplification.

Intracellular Amplification: The central innovation of the INgen method is the ability to amplify genomic DNA directly within fixed and permeabilized cells. Subsequent steps, including the use of fluorescent technologies, biotinylation, DNA sequencing, etc. are purely optional downstream steps in INgen. By performing amplification intracellularly, biases inherent in traditional single-cell sequencing workflows are mitigated, such as uneven amplification or the loss of fragile genomic regions during extraction. Instead, the INgen method leverages the cellular environment itself as a natural reaction vessel, enabling robust, contained, and targeted DNA synthesis.

The selection of an appropriate DNA polymerase was critical to the success of this approach. phi29 DNA polymerase was employed, a highly processive, strand-displacing enzyme optimized for isothermal amplification and capable of functioning despite the presence of residual fixative or membrane components. This enzyme supported consistent amplification across a variety of cell types, including yeast and bacteria. However, the INgen method is not limited to phi29 DNA polymerase, and any other polymerase(s), especially isothermal polymerase(s), may be employed with INgen.

The heart of the INgen method is the controlled intracellular reaction setup. Fixed and permeabilized cells were first incubated with a reaction mixture containing 1×phi29 buffer, 0.2 mg/mL BSA, 250 μM dNTPs, 1.15 M sorbitol, and 2 μM biotinylated primers (either gene-specific or random hexamers). Approximately 5 μL of a 1×10⁶ cells/mL suspension (~5×10³ cells total) is added to 45 μL of reaction mixture to yield a final volume of 50 μL. The mixture is pre-incubated at 72° C. for 5 minutes to facilitate primer annealing while preserving cell structure. Phi29 polymerase is then added (final concentration: 100 U per 50 μL reaction), and the complete reaction is incubated at 30° C. for 16 hours under isothermal conditions.

By avoiding traditional thermal cycling, INgen reduces membrane disruption, minimizes nonspecific extracellular amplification, and decreases the risk of amplification artifacts or false-positive signals. Amplification products remain confined within the cell and covalently bound to the (optionally used) biotinylated primers, enabling their (optional) selective purification using (optional) streptavidin-coated magnetic beads. This approach preserves genomic integrity, supports uniform genome-wide amplification, and improves the detection of low-abundance genetic features such as rare mutations or transposable elements.

Identifying Highly Amplified Cells: EvaGreen® Staining for Genome Quantification and Sorting: To quantify intracellular genome amplification, cells were stained with EvaGreen®, a DNA-binding dye that fluoresces upon binding to double-stranded DNA. Cells were incubated with EvaGreen® for 30 minutes to ensure sufficient dye uptake while minimizing background fluorescence. Following incubation, excess dye was removed by centrifugation, and the cells were washed to prevent non-specific staining.

Fluorescence intensity was measured using an Attune flow cytometer, allowing for precise quantification of amplified DNA within individual cells. Cells exhibiting high fluorescence-indicative of robust genome amplification-were identified and selected for sorting. Using fluorescence-activated cell sorting (FACS), highly amplified cells were isolated and deposited into individual wells of a 96-well plate for downstream processing. This approach enabled the targeted recovery of cells with successful intracellular amplification, improving the efficiency and accuracy of genomic analysis.

DNA Recovery and Sequencing Preparation: Capturing and Amplifying Biotinylated Genomes: Following intracellular amplification, cells were lysed using proteinase K to release genomic material. To inactivate the proteinase K, 5 μL of 100 μM PMSF (resuspended in isopropanol) was added to each tube, followed by a 10-minute incubation at room temperature. No high temperature step was included after lysis. Genomic DNA within fixed cells remains largely insoluble under these lysis conditions (non-ionic detergent, Proteinase K), and is not expected to be released into the supernatant unless the cells are physically or enzymatically disrupted.

For (optional) selective capture of intracellularly amplified, (optionally) biotinylated DNA, 100 μL of resuspended streptavidin-coated C1 magnetic beads were (optionally) added to each sample and incubated at room temperature for 60 minutes with agitation. The samples were then placed on a magnetic rack, and the supernatant was removed. Beads were resuspended in 250 μL of 1×wash solution, agitated for 5 minutes, and subjected to two consecutive wash steps. After the final wash, beads were resuspended in 250 μL of 10 mM Tris-HCl (pH 8.0) with 0.1% Tween-20. At this stage, beads could either be rinsed with molecular-grade water and processed immediately or stored overnight at 4° C. in a Tris-Tween buffer. Amplification off-the-beads was performed by adding bead bound DNA to a 50 μl reaction mix which contained 1×phi29 buffer, 0.2 mg/mL BSA, 250 UM dNTPs, 1.15 M sorbitol, and 2 μM random hexamers. Libraries were constructed using the Genomic DNA by ligation kit (SQK-LSK109) following manufacturer's instructions. Libraries were quantified using Qubit and sequencing was performed either on MinION using a FLO-MIN106D flow cell, or was prepared for Illumina sequencing, using the Illumina DNA Prep library preparation kit.

Example Fixation and Permeabilization Permutations: a) Overnight Formaldehyde Fixation with Zymolyase and Tween-20 Permeabilization. At the time of sampling, 3 mL of yeast cultures, grown to saturation in YPD medium, were immediately spun down in a room-temperature centrifuge at 5000 g for 3 minutes. The cell pellet was fixed with 4% formaldehyde and incubated overnight at +4° C. on a shaker. Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. Permeabilization was performed with 0.1% Tween-20 at room temperature for 10 minutes before proceeding to downstream steps.

b) Overnight Formaldehyde Fixation with Zymolyase and TritonX-100 Permeabilization. At the time of sampling, 3 mL of yeast cultures, grown to saturation in YPD medium, were immediately spun down in a room-temperature centrifuge at 5000 g for 3 minutes. The cell pellet was fixed with 4% formaldehyde and incubated overnight at +4° C. on a shaker. Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. Permeabilization was performed with 0.1% TritonX-100 at room temperature for 10 minutes before proceeding to downstream steps.

c) 10 Minute Formaldehyde Fixation with Zymolyase and TritonX-100 Permeabilization (“INgen fix”). At the time of sampling, 3 mL of yeast cultures, grown to saturation in YPD medium, were immediately spun down in a room-temperature centrifuge at 5000 g for 3 minutes. The cell pellet was fixed with 4% formaldehyde for 10 minutes at room temperature (+20° C.). Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. Permeabilization was performed with 0.1% TritonX-100 at room temperature for 10 minutes before proceeding to downstream steps.

d) 30 Minute Formaldehyde Fixation with Zymolyase and Overnight Ethanol Permeabilization (“FISH fix”). At the time of sampling, 3 mL of yeast cultures, grown to saturation in YPD medium, were immediately spun down in a room-temperature centrifuge at 5000 g for 3 minutes. The cell pellet was fixed with 4% formaldehyde for 30 minutes at room temperature (+20° C.) and then transferred to +4° C. for overnight fixation on a shaker. Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. The permeabilization step used ethanol treatment overnight at room temperature before downstream steps.

e) Ethanol Fixation and Permeabilization. At the time of sampling, 3 mL of Escherichia coli cultures, grown to the desired density, were immediately spun down in a room-temperature centrifuge at 5000 g for 3 minutes. The cell pellet was resuspended in 70% ethanol and incubated at room temperature (+20° C.) for 10 minutes. Fixed cells were centrifuged again at 5000 g for 3 minutes, the supernatant was discarded, and the pellet was resuspended in ice-cold PBS. This washing step was repeated twice to ensure the removal of excess ethanol. Fixed cells were then processed directly for intracellular amplification or other downstream applications.

### Additional Methods Considerations

Yeast cell culture. S. cerevisiae C5W4 WTC-SOD1-D102S was used. Cells were streaked on YP plus 2% dextrose agar plates from frozen −80° C. glycerol stocks and grown at 30° C. for 48 hours. Single colonies of a given yeast strain were inoculated into YP plus 2% dextrose liquid media (YPD) and grown at 30° C. with shaking [200 rotations per minute (rpm)] for 24 hours.

Bacterial cell culture. E. coli DH5a was used. Cells were streaked on LB agar plates from frozen −80° C. glycerol stocks and grown at 37° C. for 24 hours. Single colonies of DH5α were inoculated into fresh LB medium and grown at 37° C. with shaking [200 rotations per minute (rpm)] for 24 hours.

Cell Lysis. After amplification, the entire reaction volume was transferred into a microcentrifuge tube and 1 mL of cold PBS and 5 μL of 10% Triton X-100 was added. Samples were spun down at 4° C., 5000 g for 3 min. The supernatant was carefully aspirated off, leaving ~30 μL to avoid removing the pellet. Cells were then resuspended in 1 mL cold PBS, spun down, and resuspended in 1 mL cold PBS for a total of two washes. After washing cells, the supernatant was aspirated off and cells were resuspended in 50 μL of cold PBS, 50 μL of 2×lysis buffer [20 mM Tris (pH 8.0), 400 mM NaCl, 100 mM EDTA (pH 8.0), and 4.4% SDS], and 10 μL of proteinase K solution (20 mg/mL). Cells were incubated at 55° C. for 2 hours with periodic vortexing to lyse the cells and reverse the formaldehyde cross-links.

Preparing Streptavidin Beads for Sample Binding. To prepare beads for (optional) sample binding, they must first be washed. For each lysate, 44 μL of Dynabeads MyOne Streptavidin C1 (Invitrogen) were washed three times with 800 μL of a 1×wash solution of 5 mM Tris-HCl pH 8.0, 1 M NaCl, 500 μM EDTA, and 0.05% Tween-20 using a magnetic 1.5 mL tube rack. The beads were then resuspended in 100 μL per sample of a 2×wash solution containing 10 mM Tris-HCl PH 8.0, 2 M NaCl, and 1 mM EDTA.

Sample Binding to Streptavidin Beads. Directly after lysates were removed from heat, 5 μL of 100 μM PMSF (resuspended in isopropanol) was added to each tube and incubated at room temperature for 10 minutes to inactivate the proteinase K. To (optionally) bind DNA to C1 beads, 100 μL of resuspended C1 beads were added to each sample tube and agitated at room temperature for 60 minutes. The samples were then placed on the magnetic rack and the supernatant was removed. Samples were removed from the magnetic rack and resuspended in 250 μL of the 1×wash solution and agitated at room temperature for 5 min. Samples were replaced on the magnetic rack and the subsequent steps were repeated for a total of two wash steps. After the two 1×wash steps, the supernatant was removed, and the samples were resuspended in 250 μL of 10 mM Tris-HCl pH 8.0 and 0.1% Tween-20. At this point, the beads could be rinsed with 250 uL of molecular-grade water while the beads were still bound to the magnetic rack and moved on to subsequent steps or resuspended in 250 μL of the Tris-HCL Tween-20 buffer and stored at 4° C. overnight.

Off-the-beads Amplification. If the samples were stored overnight in the Tris-Tween buffer, the tubes were placed in a magnetic rack and the beads were rinsed with 250 μL of molecular-grade water. The bead-bound DNA was then amplified in 220 μL reactions with 2×high fidelity polymerase (KAPA HiFi) and 0.4 μM forward and reverse primers for 3 min at 95° C., and then five cycles of 98° C. for 20 s, 65° C. for 45 s, and 72 for 3 min.

Quantitative PCR. The off-the-beads PCR product was then placed against a magnetic rack and the supernatant was transferred to new optical-grade PCR tubes with qPCR dye (EvaGreen® 20×). The samples were then amplified on a qPCR machine for a further 10-20 cycles until the amplification curves exited the log-linear phase.

Size selection bead clean up. The PCR products were then cleaned using a 0.8×SPRI size selection and eluted in 20 μL of molecular-grade water.

Gel electrophoresis. 5 μL of the product which was eluted during the bead clean-up was then run on a 2% agarose gel at 120 V for 15-20 min. A single properly amplified gene should appear as a dark band on the gel at the correct size of the gene. The genome that has been properly amplified with random hexamers should appear as a smear starting at approximately 5-7 KB and ending at approximately 300 bp on a gel.

### EMBODIMENTS

The following are non-limiting embodiments of the present invention. It is to be understood that said embodiments are not intended to limit the present invention in any way. Equivalents or substitutes are within the scope of the present invention.

Embodiment 1: A method of intracellularly amplifying DNA within a cell, comprising: fixing and permeabilizing a sample comprising cells; amplifying DNA within the fixed and permeabilized cells by incubating the cells in a solution comprising: an isothermal polymerase; dNTPs; wherein the isothermal polymerases incorporate one or more dNTPs into the amplified DNA.

Embodiment 2: A method of intracellularly amplifying DNA within a cell comprising: fixing and permeabilizing a liquid sample comprising cells; and amplifying DNA within the fixed and permeabilized cells by incubating the cells in a solution comprising: an isothermal polymerase; and fluorescent dNTPs; wherein the isothermal polymerases incorporate one or more fluorescent dNTPs into the amplified DNA, thereby producing fluorescent cells.

Embodiment 3: A method of intracellularly amplifying DNA within a cell comprising: fixing and permeabilizing a liquid sample comprising cells; and amplifying DNA within the fixed and permeabilized cells by incubating the cells in a solution comprising: an isothermal polymerase; and fluorescent DNA binding dye; wherein the fluorescent DNA binding dye binds to the amplified DNA, thereby producing fluorescent cells.

Embodiment 4: The method of any one of embodiments 1-3, wherein the isothermal polymerase is a phi29 polymerase or a Bst polymerase.

Embodiment 5: The method of any one of embodiments 1-4, wherein the solution further comprises enzymes, a buffer, and one or more nucleic acid primers.

Embodiment 6: The method of any one of embodiments 2-5, wherein the solution further comprises dNTPs.

Embodiment 7: The method of embodiment 2 or 3, wherein the solution further comprises 5-ethynyl-2′-deoxyuridine (EdU) and a fluorescent azide; wherein the method further comprises incubating the cells with a fluorescent azide for a period of time; and wherein the fluorescent azide selectively binds to the one or more EdUs, thereby producing fluorescent cells.

Embodiment 8: The method of embodiment 5, wherein the nucleic acid primers comprise biotinylated primers, sequence-specific primers or random oligonucleotide primers.

Embodiment 9: A kit comprising: fixative agents; permeabilization agent; one or more nucleic acid primers; an isothermal polymerase; 5-ethynyl-2′-deoxyuridine (EdU); and an azide bound fluorophore.

Embodiment 10: A kit comprising: fixative agents; permeabilization agent; one or more nucleic acid primers; an isothermal polymerase; and fluorescent dNTPs.

Embodiment 11: A kit comprising: fixative agents; permeabilization agent; one or more nucleic acid primers; an isothermal polymerase; and fluorescent DNA binding dye. The kit may further comprise instructions that include the methods described herein.

Embodiment 12: The kit of any one of embodiments 9-11, wherein the fixative agents comprising formaldehyde or ethanol.

Embodiment 13: The kit of any one of embodiments 9-12, wherein the permeabilization agent comprises an enzymatic treatment and/or a detergent.

Embodiment 14: The kit of embodiment 13, wherein the enzymatic treatment comprises zymolyase.

Embodiment 15: The kit of embodiment 13, wherein the detergent comprises Tween-20 or Triton X-100.

Embodiment 16: The kit of any one of embodiments 9-15, wherein nucleic acid primers comprise biotinylated primers, sequence-specific primers or random oligonucleotide primers.

Embodiment 17: A method of intracellularly amplifying DNA within a cell, comprising: fixing and permeabilizing a sample comprising cells; and amplifying DNA within the fixed and permeabilized cells by incubating the cells in a solution comprising: an isothermal polymerase.

Embodiment 18: A method of intracellularly amplifying DNA within a cell comprising: fixing and permeabilizing a liquid sample comprising cells; and amplifying DNA within the fixed and permeabilized cells by incubating the cells in a solution comprising: an isothermal polymerase.

Embodiment 19: The method of embodiment 17 or embodiment 18, wherein the isothermal polymerase is a phi29 polymerase or a Bst polymerase.

Embodiment 20: The method of any one of embodiments 17-19, wherein the solution further comprises enzymes, dNTPs, a buffer, and one or more nucleic acid primers.

Embodiment 21: The method of embodiment 20, wherein the nucleic acid primers comprise biotinylated primers, sequence-specific primers or random oligonucleotide primers.

Embodiment 22: A kit comprising: fixative agents; permeabilization agent; one or more nucleic acid primers; and an isothermal polymerase. The kit may further comprise instructions that include the methods described herein.

Embodiment 23: The kit of embodiment 22, wherein the fixative agents comprising formaldehyde or ethanol.

Embodiment 24: The kit of embodiment 22 or embodiment 23, wherein the permeabilization agent comprises an enzymatic treatment and/or a detergent.

Embodiment 25: The kit of embodiment 24, wherein the enzymatic treatment comprises zymolyase.

Embodiment 26: The kit of embodiment 24, wherein the detergent comprises Tween-20 or Triton X-100.

Embodiment 27: The kit of any one of embodiments 22-26, wherein nucleic acid primers comprise biotinylated primers, sequence-specific primers or random oligonucleotide primers.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of intracellularly amplifying DNA within a cell, comprising:
a) fixing and permeabilizing a sample comprising cells; and
b) amplifying DNA within the fixed and permeabilized cells by incubating the cells in a solution comprising:
i) an isothermal polymerase;
ii) dNTPs; and
iii) one or more modified nucleic acid primers;
wherein the isothermal polymerase incorporates the one or more dNTPs into the amplified DNA, wherein the amplified DNA comprises one or more extended modified nucleic acid primers.

2. The method of claim 1, wherein the isothermal polymerase is a phi29 polymerase or a Bst polymerase.

3. The method of claim 1, wherein the solution further comprises at least one of: enzymes or a buffer.

4. The method of claim 1, wherein the one or more modified nucleic acid primers comprise biotinylated primers.

5. The method of claim 1, wherein the one or more modified nucleic acid primers comprise one or more modified sequence-specific primers, one or more modified random oligonucleotide primers, or a combination thereof.

6. A method of intracellularly amplifying DNA within a cell comprising:
a) fixing and permeabilizing a sample comprising cells; and
b) amplifying DNA within the fixed and permeabilized cells by incubating the cells in a solution comprising:
i) an isothermal polymerase; and
ii) fluorescent dNTPs;
wherein the isothermal polymerase incorporates one or more fluorescent dNTPs into the amplified DNA, thereby producing fluorescent cells.

7. The method of claim 6, wherein the sample is a liquid sample.

8. The method of claim 6, wherein the solution further comprises a fluorescent DNA binding dye; wherein the fluorescent DNA binding dye binds to the fluorescent dNTPs, thereby producing fluorescent cells.

9. The method of claim 6, wherein the isothermal polymerase is a phi29 polymerase or a Bst polymerase.

10. The method of claim 6, wherein the solution further comprises at least one of:
enzymes, a buffer, dNTPs, or one or more nucleic acid primers.

11. The method of claim 6, wherein the fluorescent dNTPs comprise 5-ethynyl-2′-deoxyuridine (EdU).

12. The method of claim 11, wherein the solution further comprises a fluorescent azide; and wherein the method comprises incubating the cells with the fluorescent azide for a period of time; and wherein the fluorescent azide selectively binds to the EdUs, thereby producing fluorescent cells.

13. The method of claim 10, wherein at least one of the nucleic acid primers comprise at least one of: biotinylated primers, sequence-specific primers or random oligonucleotide primers.

14. A kit comprising:
a) fixative agents;
b) permeabilization agent;
c) one or more modified nucleic acid primers;
d) an isothermal polymerase; and
e) instructions comprising:
i) fixing and permeabilizing a sample comprising cells; and
ii) amplifying DNA within the fixed and permeabilized cells by incubating the cells in a solution comprising the isothermal polymerase, dNTPs, and the one or more modified nucleic acid primers.

15. The kit of claim 14, further comprising at least two of: an azide bound fluorophore, fluorescent dNTPs, or fluorescent DNA binding dye.

16. The kit of claim 14, wherein the fixative agents comprise at least one of: formaldehyde, ethanol, or isopropanol.

17. The kit of claim 14, wherein the permeabilization agent comprises at least one of: an enzymatic treatment and/or a detergent.

18. The kit of claim 17, wherein the enzymatic treatment comprises zymolyase.

19. The kit of claim 17, wherein the detergent comprises Tween-20 or Triton X-100.

20. The kit of claim 14, wherein nucleic acid primers comprise at least one of: biotinylated primers, sequence-specific primers or random oligonucleotide primers.
